(19) United States
(12) Patent Application Publication
(10) Pub. No.: US 2002/0022034 A1
(43) Pub. Date: Feb. 21, 2002

(54) Therapeutic DNA vaccination

(76) Inventors: Julianna Lisziewicz, Bethesda, MD (US); Franco Lori, Bethesda, MD (US)

Correspondence Address: The Law Offices of Valerie E. Looper, 77126 Lightfall Court, Columbia, MD, 21044, US

(21) Appl. No.: 09/863,606

(22) Filed: May 23, 2001

Related U.S. Application Data
(63) Continuation-in-part of application No. 09/153,198, filed on Sep. 15, 1998.
(60) Provisional application No. 60/058,933, filed on Sep. 15, 1997.

(51) Int. Cl. A61K 39/21; A61K 31/70; A61K 48/00

(52) U.S. Cl. 424/208.1; 514/23; 514/44

(57) ABSTRACT

A method for genetic immunization comprises administering a highly active antiretroviral therapy to control viral replication, and then administering a transcutaneous DNA vaccine.

This application is a continuation-in-part of U.S. Ser. No. 09/153,198 filed Sep. 15, 1998, which is a continuation-in-part of 60/058,933, filed Sep. 15, 1997, both which are incorporated herein as if set forth in full.

### FIELD OF THE INVENTION

The present invention relates generally to methods and compositions for delivering foreign genetic material into cells. Specifically, it relates to a technique for receptor-mediated delivery of genes to cells. A gene delivery complex compatible with a specific type of targeted cell is formed from the foreign genetic material, a vector, and optionally, a carrier. The complex is then exposed to the cells under conditions permitting receptor-mediated endocytosis, resulting in the functional uptake, or transduction, of the foreign genetic material. The method is not only useful for in vitro, but also in vivo gene delivery to antigen presenting cells, specifically described as transcutaneous gene transfer to skin Langerhans cells. This technique is particularly useful for preventive and therapeutic genetic immunization when the foreign genetic material is an immunogen such as DNA encoding a substantial portion of the antigens and particles associated with an infectious virus, and where delivery by injection is undesirable. A therapeutic genetic immunization technique includes suppression of viral replication using a drug therapy, and then administering a vaccine based on the complex.

### BACKGROUND OF THE INVENTION

The immune system for animals has two different but related responses, the cellular immune response and the humoral immune response. The cellular immune response produces T lymphocytes, which kill cells having foreign identifying markers on their surface. Cells which have such identifying markers on their surface are said to “present” an antigen, and are referred to as antigen presenting cells (APCs). In addition, T lymphocytes also stimulate the humoral response by helping B cells, the precursors of plasma cells.

The humoral immune response results in the production by plasma cells of antibodies, which act on specific molecules in solution. Antibodies (or immunoglobulins) are proteins synthesized by an animal in response to the presence of a foreign substance. They are secreted by plasma cells, which are derived from B lymphocytes (B cells). These soluble proteins are the recognition elements of the humoral immune response. Each antibody has specific affinity for the foreign substance that stimulated its synthesis. That is, the antibody has a segment or site, called an antigen binding site, which will adhere to the foreign substance. A foreign macromolecule capable of eliciting the formation of antibodies against itself is called an antigen. Proteins and polysaccharides are usually effective antigens. The specific affinity of an antibody is not for the entire macromolecular antigen, but for a particular site on it called the antigenic determinant or epitope. Antibodies recognize foreign molecules in solution and on membranes irrespective of the molecule's context. The humoral immune response is most effective in combating bacteria and viruses in extracellular media. (The word humor is the Latin word for fluid or liquid.) One strategy for conferring immunity against disease is to expose the individual to one or more antigens associated with a virus or bacterium rather than use the actual virus or bacterium. Such a vaccine is known as a subunit vaccine, and it works particularly well to stimulate the production of antibodies.

T cells mediate the cellular immune response. In contrast to the humoral immune response, the cellular immune response destroys virus-infected cells, parasites, and cancer cells. The surface of T cells contain transmembrane proteins called T cell receptors that recognize foreign molecules on the surface of other cells. That is, T cells recognize antigen presenting cells (APCs). T cell receptors do not recognize isolated foreign molecules. The foreign unit must be located on the surface of a cell, and must be presented to the T cell by a particular membrane protein, one encoded by a highly variable chromosomal region of the host known as the major histocompatibility complex (MHC). The MHC encodes three classes of transmembrane proteins. MHC Class I proteins, which are expressed in nearly all types of cells, present foreign epitopes to cytotoxic T cells. MHC Class II proteins, which are expressed in immune system cells and phagocytes, present foreign epitopes to helper T cells. MHC Class III proteins are components of the process know as the complement cascade.

There are a variety of T cells, including cytoxic T lymphocytes (CTL, or killer T cells) which destroy cells that display a foreign epitope bound to an MHC protein. When the foreign-epitope-plus-MHC-protein binds to the T cell receptor, the T cell secretes granules containing perforin, which polymerizes to form transmembrane pores, thereby breaking the cell open, or inducing cell lysis. Other classes of T cells, called Helper T cells, secrete peptides and proteins called lymphokines. These hormone-like molecules direct the movements and activities of other cells. Some examples are Interleukin-2 (IL2), Interleukin-4 (IL-4), Interferons, Granulocyte-Macrophage colony-stimulating factor (GM-CSF), and Tumor necrosis Factor (TNF). The T cells are implicated in the complement cascade, a precisely regulated, complex series of events which results in the destruction of microorganisms and infected cells. More than fifteen soluble proteins co-operate to form multi-unit antigen-antibody complexes that precede the formation of large holes in the cells' plasma membrane.

Expression of foreign genes in antigen presenting cells (APC) may be used to generate efficient CTL response in animals. Therefore, gene transfer and genetic modification of APC has potential to generate effective vaccine and therapeutic approaches against different diseases, including viral infections and cancer. Live recombinant virus vectors expressing various foreign antigens, such as pox viruses, adenoviruses, and retroviruses, can be used to elicit both humoral and cellular immune response by mimicking viral infection. Also, live attenuated (or, weakened) viruses have been proposed as vaccines. DNA vaccination strategy is also being explored. Different viral genes have been cloned into plasmid DNA and injected into muscles, skin, or subcutaneously. These constructs are able to express proteins and elicit both a cellular and humoral immune response.

It has been suggested that viral diseases may be responsive to the technique of genetic immunization. Certain cells, such as dendritic cells, are known to pick up antigens and migrate from the tissues of the body to the lymphoid tissues. There these cells present the antigens in the lymphoid organs: that is, they display a foreign epitope bound to an MHC protein. Such antigen-presenting cells (APCs) are a known part of the immune response mechanism. If cells such as a dendritic cells (DC) are modified so that they contain DNA encoding a virus which is infectious but incapable of efficient reproduction, they could not only present antigens in the classic sense, but also be manipulated to produce, or express, viral particles and a wide variety of viral proteins. A novel technology has been described in Attorney Docket No. RGT-100A, U.S. Ser. No. 08/803,484 “Methods and Compositions for Protective and Therapeutic Genetic Immunization” which is incorporated herein by reference as if set forth in full. It discloses that genes of a replication-incompetent virus can be incorporated into antigen presenting cells which then migrate to the lymphoid organs and produce the full complement of viral antigens and viral particles, thereby triggering both humoral and cellular immune responses. It teaches that DC in the lymphoid organs may then express all viral antigens and produce “authentic looking” viral particles. These viral particles would therefore play a pivotal role in the generation of additional immune responses.

This reference describes in Example 13 “in vivo transduction” of cells including APC. In that example, several well-known methods including viral and non-viral gene delivery are exemplified. In Example 14 “in vivo transduction” of cells including APC are described. These utilize (1) direct DNA injection; (2) injection of liposomes or virosomes containing the DNA; (3) direct intersplenic injection of Class 4 pox viruses; and (4) rectal and vaginal suppositories carrying gene delivery vehicles. However, this reference did not describe in detail the methods of in vitro and in vivo gene delivery. That is the subject of the present invention.

There is some evidence suggesting that genetic modification of APC will be effective to vaccinate both neonatal and adult animals and humans. Ridge et al. (Science 271: 1723-1726, 1996) have injected DC expressing a foreign antigen isolated from another animal intravenously into mice. Both neonatal and adult mice injected with these DC were able to generate good CTL killing of target cells. These experiments also demonstrated that DC expressing a foreign antigen can induce protective cell-mediated immune response which is able to eliminate infected cells in case of viral infections. In addition, these experiments demonstrated that DC-mediated immunization of neonates may be possible. These experiments did not use genetically modified cells, nor did they utilize several foreign antigens nor a virus as described in the present invention.

Sarzotti, et al. (Science 271: 1726-128, 1996) demonstrated that low dose inoculation with viruses results in a protective immune response (Th1-type) which generates CTL response but high dose inoculation will result in a nonprotective (Th2-type) immune response which mainly generates antibodies. These CTL responses were very long lasting and also could be generated in neonates. High doses of virus might overwhelm and disarm T-cells before DC could activate the T-cells. Again, the route of administration, not through injection but through presentation by DC, is important. These findings are consistent with other results showing that exposure to low dose viruses provokes predominantly cellular (Th1-type) immune response. In macaques, a low dose SIV primed the Th1-type response without antibody production and protected animals against high dose challenge (Clerici et al. AIDS 8: 1391-1395, 1994). In humans, similar results were demonstrated by Rowland-Jones et al. (Nature Med 1: 59-64, 1995)

The process of modification of cells so that they contain foreign genetic material is called gene transfer, transfection or transduction. None of the papers cited herein have presented evidence of efficient gene transfer to antigen presenting cells, either in vitro or in vivo. As background for gene transfer into antigen presenting cells such as DC, several “low efficient” in vitro methods have been described, including liposome-mediated gene transfer; electroporation and retrovirus-vector- and adenovirus-vector-mediated gene transfers (Arthur, J.F et al. Cancer Gene Therapy. 4:1 17-21, 1997, Song, E. S. et al. Proc Natl Acad Sci U S A 94:5, 1943-8,1997). All of these in vitro methods involve the isolation of large populations of cells which are treated in the laboratory with a gene delivery vehicle. All human or animal applications involve the reintroduction of these genetically modified cells. Therefore, in vitro gene delivery methods are not feasible for vaccination or treatment of large numbers of individuals. Known in vivo methods include intradernal or intramuscular injection of recombinant virus vectors and intradermal, subcutaneous and intramuscular injection of plasmid DNA. None of these methods have been shown to effectively deliver genes into antigen presenting cells, such as dendritic cells, much less delivery of genes through the skin into the Langerhans cells.

### BRIEF DESCRIPTION OF THE DRAWINGS

FIG. 1 illustrates antibody mediated gene delivery into cells expressing Fc-receptors.

FIG. 2 illustrates gene delivery into dendritic cells and Langerhans cells via the mannose-receptor using PEI-man-DNA complex

FIG. 3. illustrates the transcutaneous gene delivery approach

FIG. 4 compares effectiveness of in vitro transfection of human DC using two different complexes of the present invention.

FIG. 5 CTL assay, illustrates that transduced DC are able to generate cytotoxic T cells from naive T cells: compares % cytotoxicity of DC transfected with integrase-HIV plasmid to that of control DC.

FIG. 6 illustrates the CTL response after ex vivo genetic immunization compares CTL response obtained in vivo using transfected DC.

FIG. 7. FACS analysis of cells emigrating from the skin. Plasmid DNA encoding the Green Fluorescent Protein (pGFP) was used as a reporter gene: 7a) PEIm/DNA complex applied on the surface of the skin; 7b) Control skin; 7c) PEIm/DNA complex injected subcutaneously; 7d) FITC-dextran injected subcutaneously.

FIG. 8. DNA-modified cells in the lymph node of mice after transcutaneous DNA immunization: 8a) Transduced cells expressing plasmid DNA-derived gene entering into the lymph node detected by in situ hybridization (white silver grains over the cells) labeled by the antisense Neo probe; 8b) Enlargement of FIG. 8a; 8c) Immunohistochemical staining of a lymph node to detect protein expressing cells.

FIG. 9. Dendritic cells expressed the plasmid DNA in a macaque's lymph node following transcutaneous DNA immunization: 9a) In situ hybridization dark-field microscopic image of cells showing (white) silver grains over positive mononuclear cells at the is periphery of a lymph node; 9b) A single DNA expressing cell stained with p55 (brown) that is a marker for lymph node DC. The black dots are silver grains (in situ hybridization) demonstrating the expression of the foreign gene.

FIG. 10. Immunogenicity of DermaVirSHIV transcutaneous DNA immunization: Representative histograms (macaque #1) to illustrate the detection of Virus-specific Immune Responses (VIR) measured as IFN-g expression by CD8+, CD3 gated T lymphocytes (CD8VIR). Left panels: percentage of CD8+, IFN-g+ T lymphocytes, CD3 gated, in the absence of antigenic stimulation (background); central panels: after stimulation with an unspecific antigen (HIV); right panels: after SIV stimulation. Numbers are the percentages of CD3+ cells in the quadrates. Upper panels illustrate results obtained before;, lower panel after transcutaneuous immunization.

FIG. 11. Median viral load and CD4 counts during HAART (11a) and STI-HAART (11b) treatment of SIV251-infected rhesus macaques with AIDS. Monkeys were treated with d(R)-9-(2-Phosphonylmethoxypropyl) adenine, didanosine and hydroxyurea (“HAART”) during the indicated time, as described previously 1. Symbols: triangles, CD4 counts; squares, viral load.

FIG. 12. DermaVirSHIV vaccination in combination with STI-HAART for the reatment of SIV251-infected macaques with AIDS. Treatment schedule and median viral load or the cohort before (12a) and after (12b) the initiation of therapeutic vaccination.

FIG. 13. Virological and immunological characterization of monkey #51 (13a), #56(13b), #60 (13c) treated with STI-HAART and DermaVirSHIV. Treatment: dotted line (details see FIG. 12b). Symbols: squares, viral load; triangles, CD4 counts.

FIG. 14. Comparison of the viral load rebound rates during treatment interruptions between acutely infected and late stage AIDS monkeys treated with STI-HAART and STI-HAART-DermaVirSHIV.

### DETAILED DESCRIPTION OF THE DRAWINGS

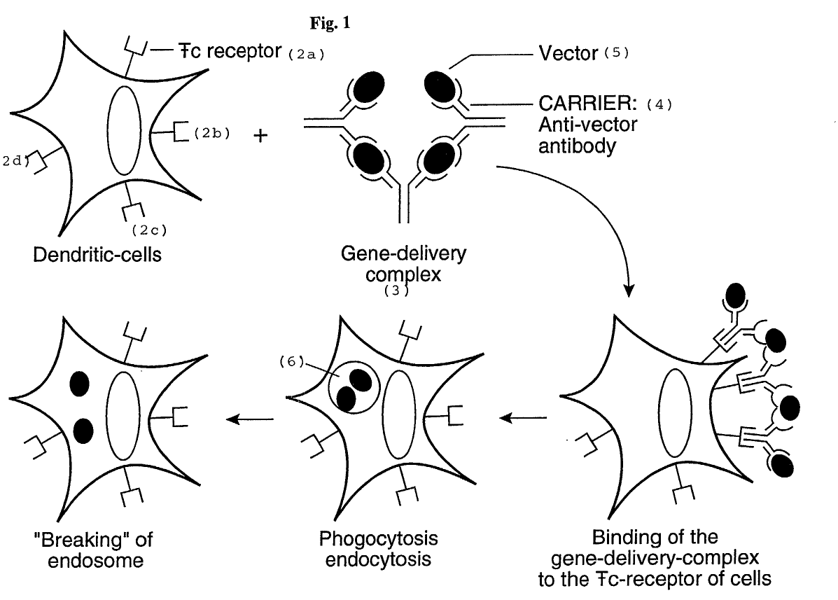

In FIG. 1, the process for antibody mediated gene delivery into cells expressing Fc-receptors is illustrated conceptually. Target cells (1) having one or more receptors (2 a,b,c,d) are exposed to a gene-delivery complex (3) comprising a carrier (4) and a vector (5) which includes the foreign genetic material. The gene delivery complex (3) binds to the receptors (2 a,b,c,d) of the cell (1) and the vector (4) is incorporated into the cell via endocytosis or phagocytosis in an endosome (6). The vector (4) has the property of breaking the endosome (6), allowing the foreign genetic material to be released into the cell.

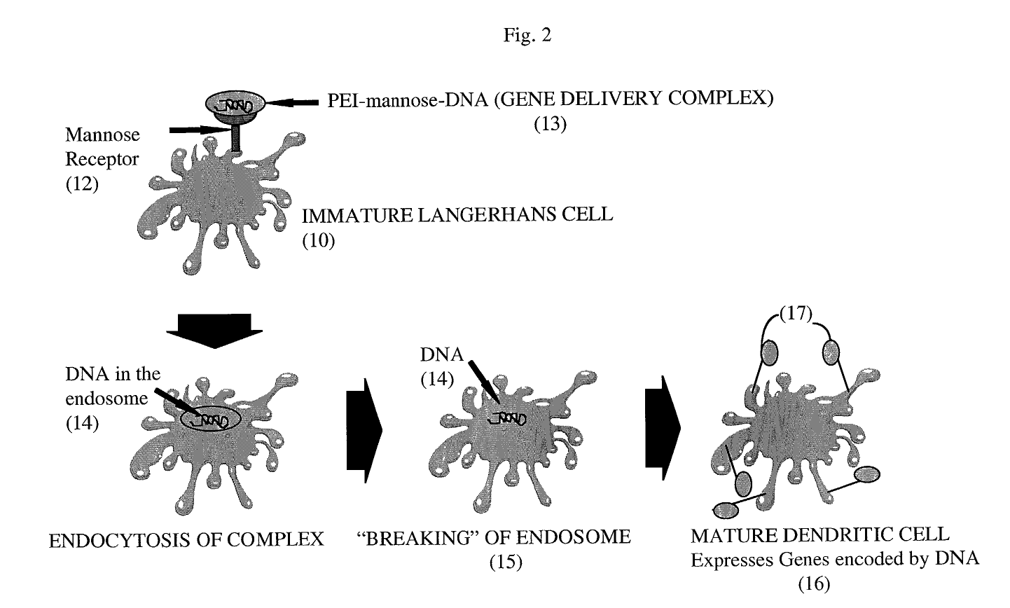

In FIG. 2, the process for sugar-mediated gene delivery into cells expressing mannose-receptors is illustrated conceptually. Target cells (10), in this case, immature Langerhans cells having one or more mannose-receptors (12) are exposed to a gene-delivery complex (13) comprising a polyethylenimine-sugar (mannose) complexed with the foreign genetic material. The gene delivery complex (13) binds to the receptors (12) of the cell (10) and the PEI-man-DNA is incorporated into the cell via endocytosis in an endosome (14). The vector (PEI-man) has the property of breaking (15) the endosome , allowing the foreign genetic material to be released into the cell. The cell matures (16) and expresses proteins (17) coded by the foreign genetic material.

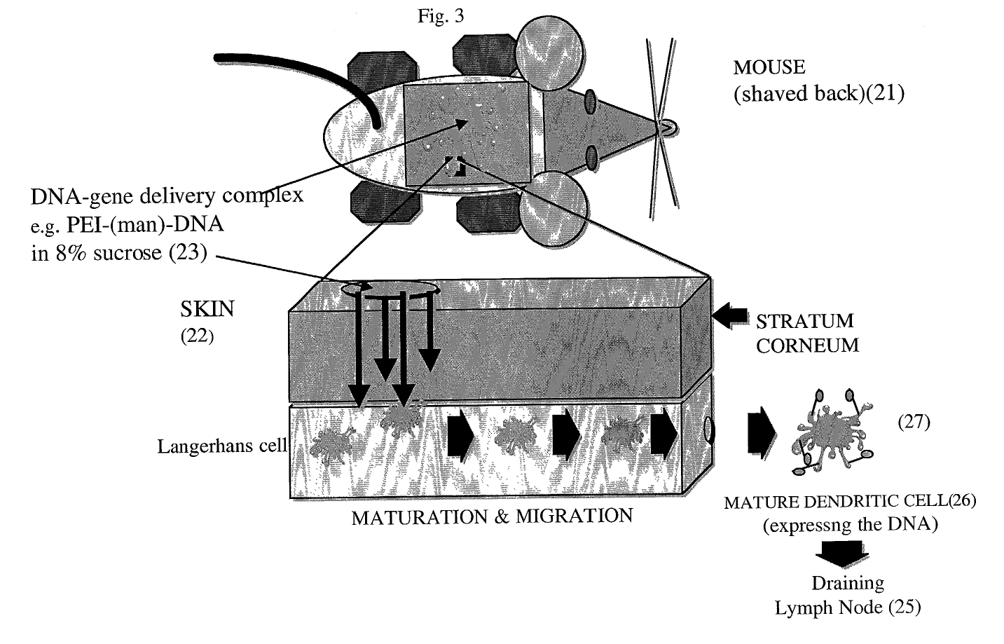

In FIG. 3, the experiment demonstrating in vivo the sugar-mediated gene delivery into cells expressing mannose-receptors is illustrated. Target cells are Langerhans cells in the skin known to express mannose receptors. Mice (21) were anesthetized and an area on the back of each mouse (22) was shaved. The shaved surface was cleaned with ethanol. PEI-man-DNA gene delivery complex in 8% glucose (23) was applied to the shaved area (22) of each mouse. Langerhans cells (24) found in the shaved area of the skin (22) pick up the complex as described in FIG. 2 above, get activated and migrate (24) to the draining lymph node (25). During the migration Langerhans cells (24) mature to be dendritic cells (26) and express the protein (27) encoded by the DNA.

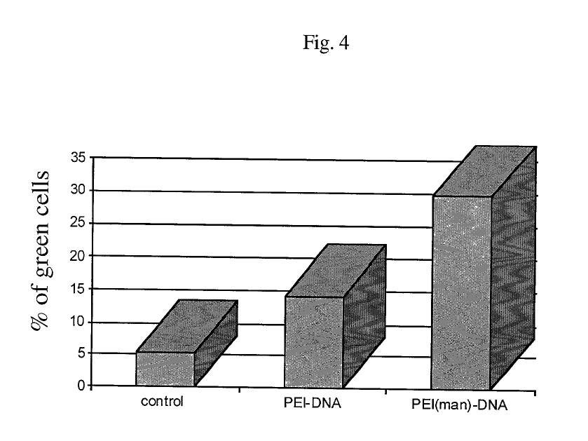

In FIG. 4, experimental results are demonstrating the in vitro gene delivery with PEI-DNA vs. PEI-man-DNA complexes. Human DC were cultured as described in the text and transfected with complexes. A marker gene encoding a green fluorescent protein (GFP) was used as the DNA. In these experiments the complexes were dissolved in a solution of NaCl. The experiment demonstrated that PEI-man is more efficient to transfect cultured DC than PEI.

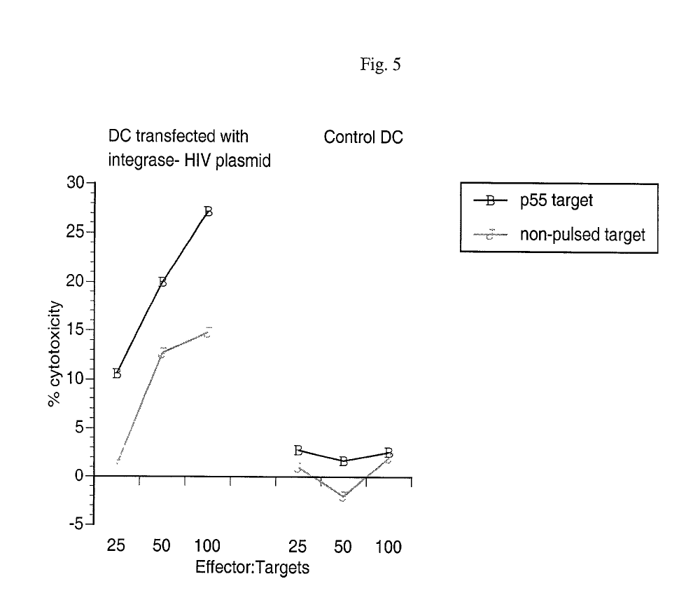

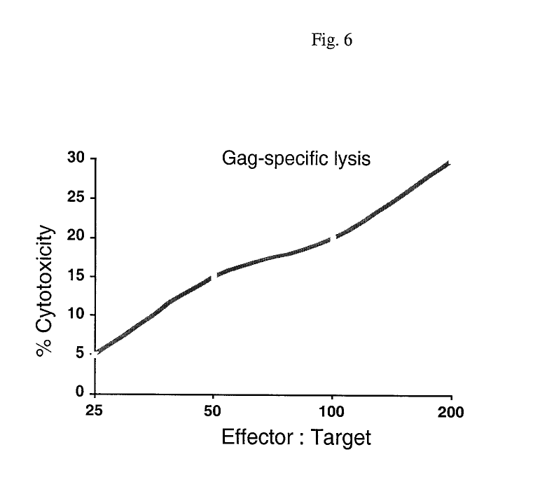

FIGS. 5-6 report experimental results and are discussed in detail in the Example section, below.

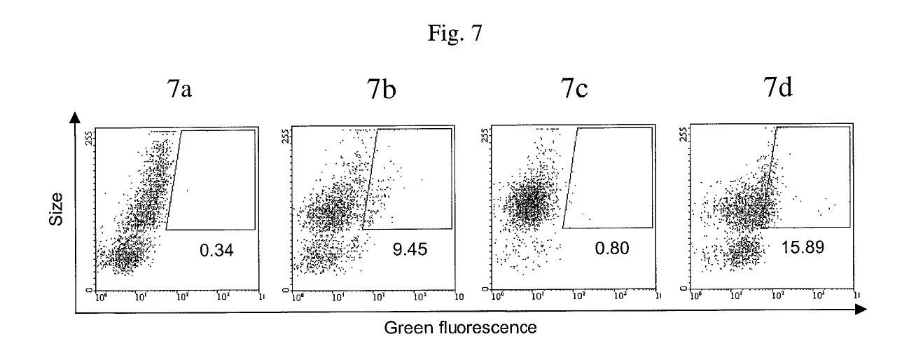

FIG. 7. FACS analysis of cells emigrating from the skin. Plasmid DNA encoding the Green Fluorescent Protein (pGFP) was used as a reporter gene: 7a) PEIm/DNA complex applied on the surface of the skin; 7b) Control skin; 7c) PEIm/DNA complex injected subcutaneously; 7d) FITC-dextran injected subcutaneously.

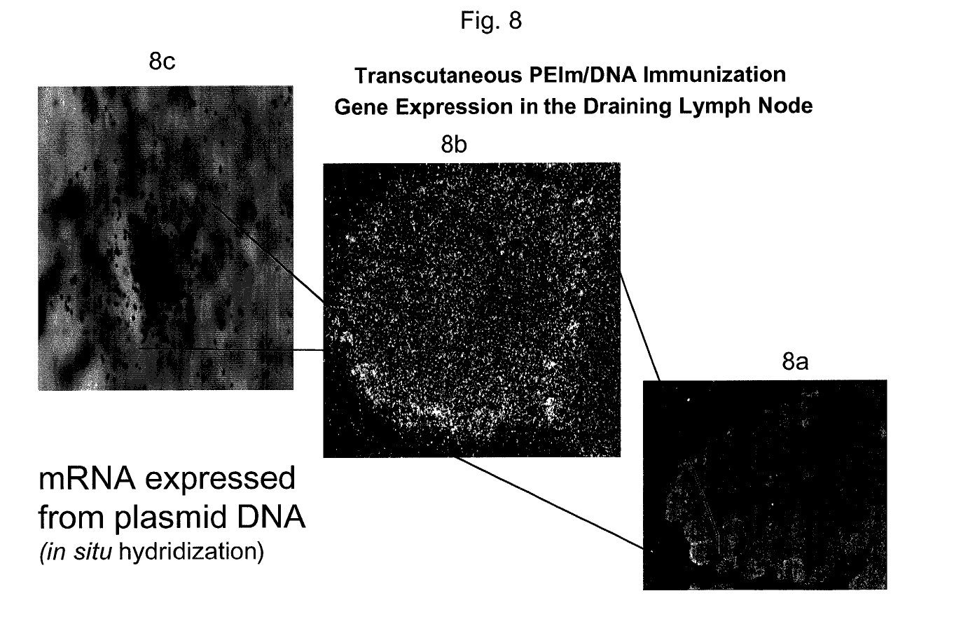

FIG. 8. DNA-modified cells in the lymph node of mice after transcutaneous DNA immunization: 8a) Transduced cells expressing plasmid DNA-derived gene entering into the lymph node detected by in situ hybridization (white silver grains over the cells) labeled by the antisense Neo probe; 8b) Enlargement of FIG. 8a; 8c) Immunohistochemical staining of a lymph node to detect protein expressing cells.

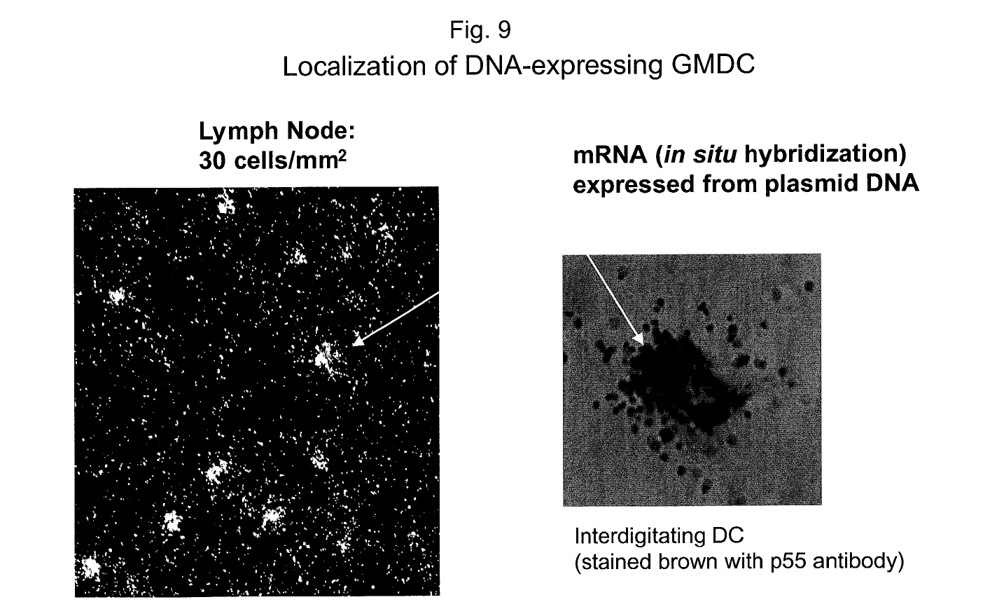

FIG. 9. Dendritic cells expressed the plasmid DNA in a macaque's lymph node following transcutaneous DNA immunization: 9a) In situ hybridization dark-field microscopic image of cells showing (white) silver grains over positive mononuclear cells at the periphery of a lymph node; 9b) A single DNA expressing cell stained with p55 (brown) that is a marker for lymph node DC. The black dots are silver grains (in situ hybridization) demonstrating the expression of the foreign gene.

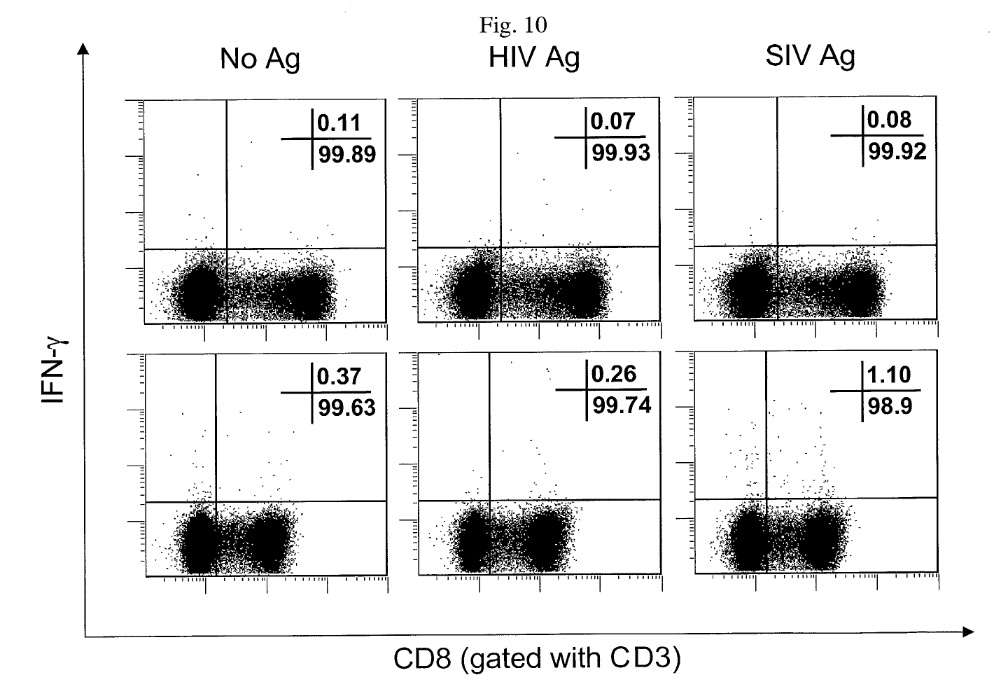

FIG. 10. Immunogenicity of DermaVirSHIV transcutaneous DNA immunization. Representative histograms (macaque #1) to illustrate the detection of Virus-specific Immune Responses (VIR) measured as IFN-g expression by CD8+, CD3 gated T lymphocytes (CD8VIR). Left panels: percentage of CD8+, IFN-g+ T lymphocytes, CD3 gated, in the absence of antigenic stimulation (background); central panels: after stimulation with an unspecific antigen (HIV); right panels: after SIV stimulation. Numbers are the percentages of CD3+ cells in the quadrates. Upper panels illustrate results obtained before; lower panel after transcutaneuous immunization.

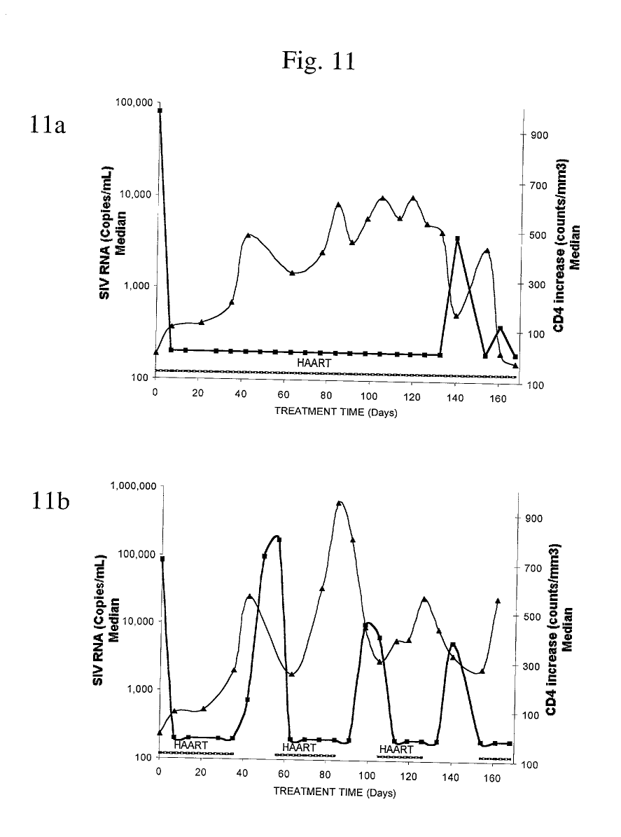

FIG. 11. Median viral load and CD4 counts during HAART (11a) and STI-HAART (11b) treatment of SIV251-infected rhesus macaques with AIDS. Monkeys were treated with d(R)-9-(2-Phosphonylmethoxypropyl) adenine, didanosine and hydroxyurea (“HAART”) during the indicated time, as described previously. Symbols: triangles, CD4 counts; squares, viral load.

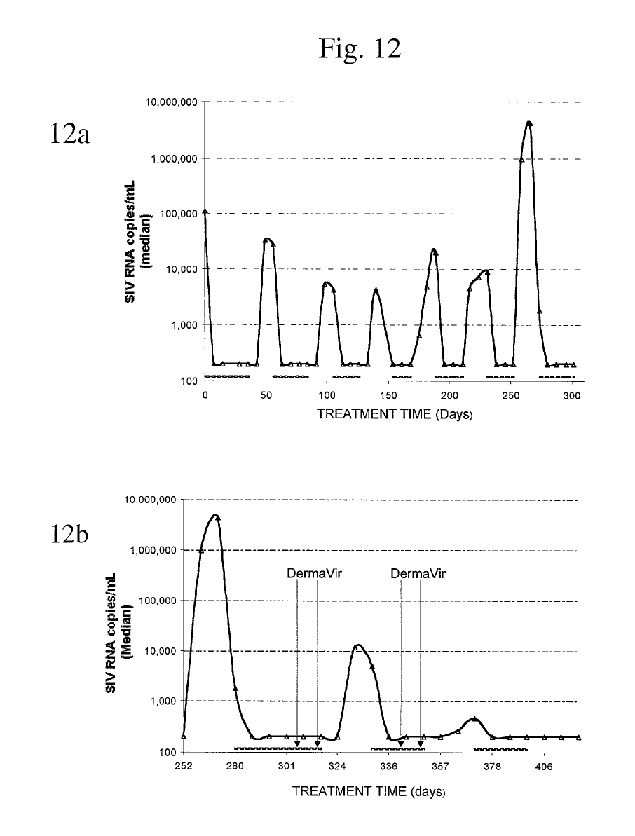

FIG. 12. DermaVirSHIV vaccination in combination with STI-HAART for the treatment of SIV251-infected macaques with AIDS. Treatment schedule and median viral load of the cohort before (12a) and after (12b) the initiation of therapeutic vaccination.

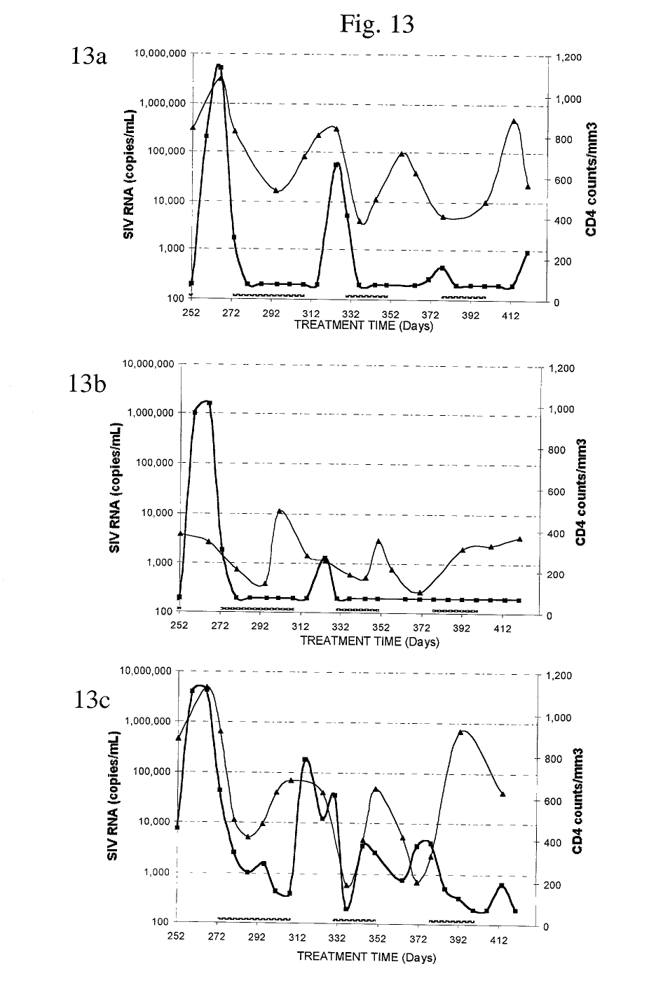

FIG. 13. Virological and immunological characterization of monkey #51 (13a), #56(13b), #60 (13c) treated with STI-HAART and DermaVirSHIV. Treatment: dotted line (details see FIG. 12b). Symbols: squares, viral load; triangles, CD4 counts.

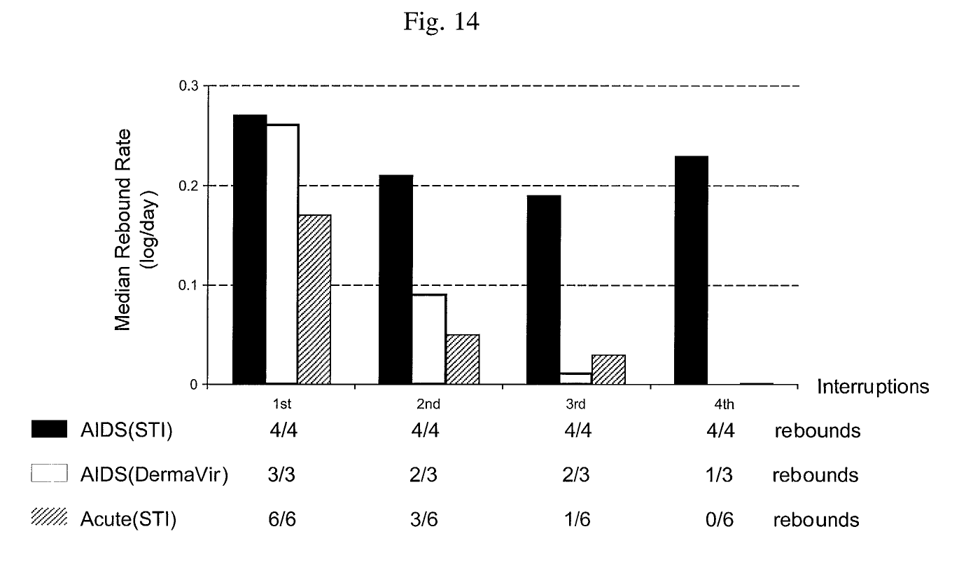

FIG. 14. Comparison of the viral load rebound rates during treatment interruptions between acutely infected and late stage AIDS monkeys treated with STI-HAART and STI-HAART-DermaVirSHIV.

### SUMMARY OF THE INVENTION

It is an object of the present invention to provide a method of improved efficiency of gene transfer to cells, in vitro and in vivo. A further object of this invention is to provide an improved method of genetic immunization by increasing the efficiency of gene transfer to antigen presenting cells. It is yet a further object of this invention to provide a means of stimulating both humoral and cellular immune responses to the protein product of the transferred genetic material. Yet another object of this invention is to provide an effective immune response to viral diseases. Yet another object of this invention is to provide a vaccine for viral diseases which is effective and has improved safety. Another object of this invention is to provide a practical, non-invasive transcutaneous gene transfer technology that can be used to genetically engineer large numbers of lymph node dendritic cells in order to induce potent T cell-mediated immune responses. Yet another objective of this novel technology is to provide a new, general scheme for developing therapeutic and preventive vaccines against infectious and neoplastic diseases.

An advantage of the present invention is that it provides a in vivo gene transfer method which can be utilized for immunotherapy and vaccination for a wide variety of diseases. An another advantage of the present invention is that it can utilize any type of DNA, or RNA, including plasmid DNA encoding immunogens like oncogens, immunogens (causing allergy), viral proteins or different types of replication defective viruses, defective viral particles, as well as plasmid DNA. An another advantage of the present invention is that it can utilize instead of DNA proteins like oncogenic protein (e.g. mutated p53 or Ras), immunogens (causing allergy), viral proteins or different types of replication defective viruses. Yet another advantage of this vaccine is that it can be administered transdermally, that is, by placing the formulation on the skin, without the use of needles. These and other objects and advantages of the present invention will become apparent through the text and examples herein.

The objects and advantages of the present invention are achieved by forming a gene delivery complex comprising a vector (which contains the desired foreign genetic material) and a carrier (which can bind both to the cells and to the gene delivery particle), then exposing target cells to the complex under conditions permitting endocytosis. The vector has the characteristic that it allows the genetic material to escape from endosomal degradation and it delivers the desired foreign genetic material to either the cytoplasm or to the nucleus. Foreign proteins can be expressed and presented to the immune system by the genetically modified cells. If the foreign genetic material encodes a replication defective virus as described in Attorney Docket No. RGT-100A, USSN “Methods and Compositions for Protective and Therapeutic Genetic Immunization” which is incorporated herein as if set forth in full, the altered target cells may then present viral antigens and also express viral particles and proteins in the lymphoid organs, thereby generating an effective cellular immune response as well as a humoral immune response.

In a preferred embodiment, plasmid DNA encoding one or more antigens is formulated with mannosylated polyethylenimine (PEIm) in an aqueous glucose solution, and applied to the surface of the skin, and the patient responds by raising an immune response to the antigen. The Examples herein demonstrate that the DNA transduces Langerhans cells in the epidermis. DNA-expressing Langerhans cells migrate to the T cell area of the draining lymph node, interdigitate as dendritic cells and present DNA-derived antigens to T cells.

In another preferred embodiment, an appropriate therapy such as a highly active antiretroviral therapy, is used to effectively suppress viral replication, and then the DNA formulation is administered.

### DETAILED DESCRIPTION OF THE INVENTION

The subject invention most closely concerns methods and compositions for the delivery of foreign genetic material into cells. It is particularly useful to enhance the efficiency of genetic immunization by increasing the efficiency of gene transfer to certain cells participating in the immune system, such as antigen presenting cells (APCs). Although the goal of the inventors is to deliver genetic material into cells, the inventors contemplate that any molecule of suitable size and configuration can be delivered into cells using the present invention. Thus, other materials such as drugs or proteins, for example, can be delivered to targeted cells using the techniques described herein.

The present invention takes advantage of some of the natural pathways available in animals. It is known, for example, that specific proteins are imported into cells by a process called receptor-mediated endocytosis. In this process, specific proteins, or ligands, bind to specific receptors in the plasma membrane of a cell. The membrane forms a vesicle, or pocket, around the protein and eventually internalizes the ligand. That is, it imports the protein into the cell. Afterward the endosome typically delivers these complexes to a lysosome where they are digested into their component parts, peptides. In cells where MHC expression occurs, peptide-MHC complexes accumulate in the lysosome and then reach the surface of the cell in a process called antigen presentation. Receptor-mediated endocytosis is the means of delivering large molecules such as essential metabolites, hormones and growth factors to cells. It is a pathway exploited by many viruses and toxins to gain entry into cells, and also plays a part in the immune response. For example, phagocytic cells have receptors enabling them to take up antigen-antibody complexes.

Particles complexed with antibodies such as IgG or complements such as C3b, or both, can efficiently enter into cells through receptor-mediated endocytosis and phagocytosis. Antibodies, or immunoglobulins, have different portions that allow them to perform different functions. For example, Immunoglobulin G (IgG) is a Y-shaped molecule with two F_{ab} segments having antigen binding sites and an F_{c} segment which mediates effector functions. Multivalent antigens can bind to antibodies and form immune complexes. The size of these immune complexes is a function of the relative concentration of antigen and antibody.

Endocytosis can be enhanced by a process known as opsonization. Opsonization is a process whereby antibodies coat antigens, thereby providing a means for other components of the immune system to recognize and respond to the antigens. Immunoglobulins with the appropriate F_{ab} sites can be used to coat the antigen particles, and subsequently, cells expressing the corresponding F_{c} receptors can recognize the F_{c} part of the opsonized antigens and readily endocytose them. Complements such as C3b, C4b and C3bi also have opsonization activity. Larger immune complexes are more effectively phagocytosed than small ones by cells such as B-cells, mononuclear phagocytes, granulocytes, neutrophils and dendritic cells expressing receptors for the F_{c} portions of immunoglobulin molecules.

IgG antibodies can be made in animals by injecting the antigen with or without adjuvants. Also, antibodies can be cloned and humanized using molecular genetic techniques. Other receptors that are commonly located on the membranes of immune system cells include, for example, transferrin, mannose and asialoglycoprotein receptors, which could easily be used for the transduction of immune system cells. The F_{c} receptor part of the antibody can also be replaced with other receptor-binding domains using molecular genetic techniques. For the inventors' present purpose, the F_{c} receptors and corresponding IgG molecules are convenient, as these serve the further function of transporting genes to dendritic cells.

Once a molecule or particle is taken up into a cell via endocytosis or phagocytosis, it is contained in a protein-receptor complex called an endosome. Endosomes are intracellular acidic compartments that serve a sorting function. Phagosomes, which result from phagocytosis, are large (10x-20x) endosomes. Endosomes then fuse with lysosomes where the material is digested to smaller products such as peptides, nucleotides and sugars. In the present invention, the role of the vector is to provide the foreign gene to the cell and avoid degradation of the gene. That is, the vector must be able to break the endosome and release the gene into the intracellular fluid, cytosol, or onto the nucleus. A number of particles are known to be able to break the endosome after receptor-mediated endocytosis, including viral gene delivery particles such as adenovirus vectors, retrovirus vectors, pox-virus vectors, and SV-40 virus. Non-viral gene delivery particles include conjugates of DNA with polylysine, polyethylenimine and its derivatives, liposomes, virosomes and chemicals which increase the pH in the endosome, such as chioroquine.

Target Cells

This invention can be used with any cells capable of receptor-mediated endocytosis or phagocytosis. The target cells must express a receptor site which, upon binding with a complementary molecule, can bring the desired molecule into the endosome or phagosome. For the inventor's present purpose, such cells are preferably cells which participate in the immune response. They include cells which can engage in receptor-mediated endocytosis and phagocytosis of antigens. Such cells include, for example, B-cells, mononuclear phagocytes, granulocytes and dendritic cells. These cells express receptors for the F_{c} portion of immunoglobulins or complement receptors, or both. Dendritic cells and macrophages are particularly preferred, because they can efficiently present foreign antigens, thereby provoking cellular immune response, or CTL response. The cells can also be targeted through other receptors such as the transferrin and mannose receptors.

Dendritic cells reside in the lymphoid tissues, such as the spleen, tonsils and lymph nodes, but they can be found in the blood, epidermis, mucosa, and other peripheral tissues. These cells pick up antigens and migrate with the antigens to the lymphoid tissues. In the skin, dendritic cells called Langerhans cells can be found in the epidermis. When they endocytose an antigen, they migrate into regional lymph nodes. In the lymph node they are called interdigitating cells, and they present the antigen to naive T-cells, provoking the cellular immune response.

To increase the efficiency of gene transfer, the number of available dendritic cells should be maximized. Choice of location can be a factor. High concentrations of dendritic cells are found, for example, in the skin and on mucosa, such as the mouth, vagina and rectum. Immature DC in the tissues can efficiently endocytose, therefore they are a good target of the gene delivery complex which delivers genes with receptor-mediated endocytosis. However, for efficient expression of MHC molecules and antigen presentation, DC must also be activated. In vitro, immature DC can be generated from peripheral blood with GM-CSF and IL-4 or from bone marrow precursors with GM-CFS. Activation of these immature DC can be induced in vitro and in vivo by bacterial products such as lipolysaccharid and TNF-alpha (Watts C. Nature 338: 724-725, 1997).

Dendritic cells can be attracted to a specific location and activated by an event implicating the immune system such as a cell or tissue injury. For the present purpose, attraction and activation of antigen presenting cells, including dendritic cells, can be mediated by an immune response unrelated to vaccination or viral infection. An example would be the skin rash that is the result of contact sensitivity to chemicals such as drugs and toxins, cosmetics and environmental antigens. If a chemical irritant is swabbed onto the skin, a rash or lesion will usually appear 24-48 hours after exposure. The lesion is due to neoantigens created by binding chemicals to the surface proteins of Langerhans cells. Neoantigens are covalently modified “normal” proteins (e.g. phosphorilated) which are recognized by antibodies. At this site, higher than normal amounts of dendritic cells may be found, and they are more likely to be activated, that is, more receptive to immediate endocytosis of an antigen. The choice of irritant depends on its efficacy to attract DC and on its side effects. In another embodiment of the invention, an immune complex-mediated injury can be created. In that case, immune complexes with both antigen and antibody components can be used to activate B cells and the complement cascade with resultant tissue injury.

Since the cells are targeted through a specific class of receptors, a particular advantage of this invention is that the gene delivery complex can be made to target specific cells. If the gene delivery complex is made with IgG or a polyethylenimine modified with an appropriate starch or sugar, it will be taken up mainly by antigen presenting cells. This would be a great advantage in the development of gene-based vaccines. Targeting other cells expressing, for example, complement receptors or transferrin receptors is also feasible as described above.

Gene Delivery Complex

The gene delivery complex of the present invention can be used to deliver genes in vitro or in vivo to cells carrying a given receptor. The gene delivery complex is built from two parts: the genetic material and a delivery particle, and may further comprise a carrier (See FIG. 1). In one embodiment, the genetic material is derived from an attenuated HIV virus and the delivery particle is non-viral vector.

In another embodiment, the same or different genetic material may be combined with a viral vector and a carrier. In that instance, the carrier is preferably an antibody that has a site complementing a receptor present on the surface of the target cell, and an antigen binding site specific to the desired delivery particle. Such a carrier is specific to both the cell and the delivery particle. For genetic immunization, human IgG specific to the gene delivery particle is conveniently selected. If there is no antibody commercially available, it can be made with techniques known to one of ordinary skill in the art. If the gene delivery particle is replication-defective HIV-1, human IgG can be used as a carrier, and is available in large quantities for passive immunotherapy. The gene delivery particle can be complexed with the antibody by incubating them together for 5 minutes at room temperature. The relative amounts of gene delivery particle and antibody are determined by whether it is desired to opsonize the gene delivery particles.

The Genetic Material

The genetic material, either DNA or RNA, is carried by the delivery complex. One or more genes can be encoded on a strand of plasmid DNA, on double-stranded DNA or on RNA. Alternatively, the genetic material can be built into recombinant viruses if they are used as a gene delivery particle. If the purpose of the gene transfer is to induce an immune response, then the genetic material must express one or more immunogenic proteins. Transduced cells will subsequently express enough of the immunogenic proteins (different viral antigens and produce authentic enough viral particles) to provoke a sufficient immune response (e.g., protect the individual from infection by the wild-type virus).

The choice of the gene delivery particle will be determined by the disease and the choice of gene(s) to transfer. Where it is desired to construct a vaccine for a reverse-transcriptase dependent virus such as HIV, the DNA preferably encodes at least a substantial portion of a replication-or integration-defective virus or the replication- or integration-defective virus itself. Examples include but are not limited to integrase negative mutants of a dual-tropic primary isolate such as HIV-1/LW, and derivatives thereof having a deletion in the protease cleavage site of the gag gene. In a preferred embodiment, the DNA further includes one or more stop codons in one or more of the reading frames of the integrase gene See Methods and Compositions for Protective and Therapeutic Genetic Immunity, U.S. Ser. No. 08/803,484 filed Feb. 20, 1997 and incorporated by reference as if set forth in full. Where it is desired to construct a vaccine for cancer, the immunogen is preferably DNA encoding one or more oncogens. Other DNA constructs can be DNA encoding replication defective Human Papilloma Virus (causing cervical cancer), replication defective Hepatitis A, B and C viruses (causing hepatitis and liver cancer), and DNA encoding replication defective animal viruses like Bovine Leukemia Virus or Feline Immunodeficiency Virus. Choices for a delivery particle incorporating the foreign genetic material can include: (a) replication defective HIV or other retrovirus; (b) recombinant adenovirus; (c) plasmid or linear DNA or RNA complexed with PEI or a derivative of PEI; (d) a virosome containing any DNA or RNA; (e) liposome containing DNA or RNA; (f) plasmid DNA-polylysine-virus complex; (g) sugar complexed with any DNA or RNA.

The Delivery System

In order to effectively deliver the genes to the cell, the gene delivery system must contain the gene or genes to be delivered and also must have the capacity to break the endosome (or phagosome), rather than be delivered to a lysosome or be isolated on the outside surface of a cell and targeted for destruction. Further, the gene delivery system must facilitate incorporation of the foreign genetic material into the genetic material of the cell.

The gene delivery system can include either a viral or non-viral vector. Viral gene delivery systems include recombinant virus vectors such as adenovirus vectors, retrovirus vectors, pox-virus vectors, mutant viruses (described above) and virosomes. Non-viral gene delivery systems include DNA conjugates with sugar, polylysine, polyethylenimine, polyethylenimine derivatives, and liposomes, together with their derivatives.

Non-viral gene delivery systems such as those utilizing sugars, sugar derivatives, liposomes, liposome derivatives and polyethylenimine or polyethylenimine derivatives are preferred. Of these, sugar and polyethylenimine derivatives adapted to target the mannose receptors of immune system cells are most preferred.

Non-viral gene delivery systems offer several advantages over viral gene delivery systems: 1) First, the non-viral vector is not recognized by the immune system, so no immune response is generated against it. As a result, it is more likely that individuals treated with the ultimate vaccine will tolerate and develop adequate immune response in cases of repeated immunization; 2) non-viral systems are potentially more safe that viral systems because there is no possibility that the system will mutate in an unexpected fashion; 3) non viral systems can be chemically synthesized in a large amounts, and are therefore potentially less expensive.

The preferred embodiment is based on a cationic polymer, polyethylenimine(PEI). PEI binds to DNA and makes the complex. The PEI-DNA complex can enter into the endosome of the skin's antigen presenting cells, Langerhans cells, via asialoglycoprotein receptor-mediated endocytosis. Then, the PEI component of this complex utilizes endosome buffering and swelling as an escape mechanism to the cytoplasm [Pollard H; Remy J S; Loussouarn G; Demolombe S; Behr J P; J Biol Chem Mar. 27, 1998;273(13):7507-11]. PEI can also be modified to target other receptors. For example, a PEI derivative, such as a sugar-modified PEI, obtains similar results, except that it is taken up through the cells' mannose receptor. Such derivatives can be made in the laboratory. For example, an isothiocyanantophenyl phenyl mannose derivative can be coupled to PEI 25 kDa, yielding a ligand (or, mannose residue of low affinity for the mannose receptor, 1 mM). Another possibility is to use linear PEI 22 k Da derivatized mannotenpaose ligand. (These materials were graciously supplied by Dr. Jean-Paul Behr, Laboratoire de Chimie Genetique, Faculte de Pharmacie, CNRS-UMR 7514 74 route du Rhin 67401 Illkirch, France)

The mannose receptor is a 175-kDa transmembrane glycoprotein that specifically expressed on the surface of macrophages and Langerhans cells. The ectodomain of the mannose receptor has eight carbohydrate recognition domains. The mannose receptor recognizes the patterns of sugars that adorn a wide array of bacteria, parasites, yeast, fungi, and mannosylated ligands. [Takahashi K; Donovan M J; Rogers R A; Ezekowitz R A, Cell Tissue Res 1998 May; 292(2):311-23]. In contrast to F_{c} receptor, the mannose receptor reconstitutes itself while releasing its cargo [Stahl et al. Cell 1980 19:207]. It thus can internalize of ligands in successive rounds, in a manner similar to the transferrin receptor, providing a sustained capacity for antigen capture[Goldstein, et al, 1985, Annu Rev Cell Biol. 1:1]. It has been recently discovered that mannose-receptor-mediated uptake of antigens results in about 100 fold more efficient antigen presentation to T-cells, as compared to antigens internalized via fluid phase [Engering et al. 1997, Eur. J. Immunol. 27:2417-2425]. This enhanced antigen presentation is due to highly efficient uptake of antigens via the mannose receptor. For these reasons we believed that targeting the mannose receptor may yield both specificity for antigen presenting cells and improved efficiency of functional uptake of the complex into the endosome.

The Carrier

The carrier of the present invention is the part of the gene delivery complex which joins a gene delivery system with a cellular receptor. In one embodiment the carrier is an immunoglobulin G (IgG). IgG is a Y-shaped molecule with two F_{ab} segments having antigen binding sites and an F_{c} segment which binds to the cellular receptor called F_{c}-receptor. Immune system cells such as B-cells, mononuclear phagocytes, granulocytes and dendritic cells have F_{c} receptors. When IgG is used as a carrier, it targets specifically cells having F_{c} receptors.

To change the carrier specificity to target other cells, the F_{c} part of the antibody can be replaced by other receptor binding domains, such as complement, sugar, or transferrin.

Where the carrier is an antibody large complexes are formed, the carrier and gene delivery system are preferably combined in equal proportions. Where it is desirable to opsonize the particle, the amount of the carrier greatly exceeds the amount of the gene delivery particles. Both endocytosis and phagocytosis are enhanced in the case of large complexes and opsonized particles. The gene delivery complex is preferably opsonized with the carrier. Where an opsonized gene delivery complex made of an antibody complexed with a delivery particle incorporating foreign genetic material is administered to an individual, cellular immune response will be maximized over the humoral immune response. The dendritic cells will be activated by the opsonized complexes, and endocytosis will be more efficient. Also, the multiple antibodies will block the antigenic determinants (epitopes) of the delivery particle. Therefore, no direct antibody response to the delivery particle would be expected. Also, some antibody complexed antigens will bind to the F_{c} receptor site of B cells, further inhibiting their antibody response. However, cellular immunity would be stimulated because the complex would be endocytosed or phagocytosed by various kinds of antigen presenting cells, including dendritic cells and macrophages.

In a preferred embodiment, the carrier is covalently joined to the non viral gene delivery system. PEI can be chemically modified with sugars (e.g., mannose, glucose, galactose, etc.). The carrier in this case is the sugar ligand, which is recognized by the mannose receptor. To change the carrier's specificity, sugar can be replaced by other receptor-binding domains.

In vivo Gene Delivery

The gene delivery complex can be injected directly into the blood, skin, or other place where cells corresponding to the carrier binding specificity are located. The complex can be applied on the skin or mucosa surfaces directly. In that event, it is preferable that the Langerhans' cells are activated on the surface. Activation may be achieved by receptor stimulation (e.g., mannose receptor), toxin activation (cholera toxin), a tissue or cell injury such as inflammation, and may be the consequence of another antigenic stimulation.

The complex can be infused using a pediatric feeding tube orally, vaginally or rectally in the case of human or animal adults or neonates. Neonates may respond better to oral administration than adults. Alternatively, the gene delivery complex may be packaged in a suppository and inserted in the vagina or rectum.

Where a viral delivery particle is used, the delivery particle can be injected directly into the muscle or skin, in the presence or absence of adjuvants, of the subject on two separate occasions for high titer of antibody production in vivo. The first injection will result primarily in a humoral immune response. That is, the capability to produce large numbers of antibodies will result. Where a concentration of IgG antibodies sufficient to opsonize the delivery particles is available, (it can be measured or assessed by experience) then the delivery particle can be administered a second time as described in 1-3 above. The site of the second administration must be chosen carefully to ensure that cells are present which can phagocytose or endocytose the opsonized antigens.

Treatment of Active Infection

The vaccine of the present invention might also be used as a method of treating active HIV infection. HIV replicates abundantly, mutates rapidly, and damages the immune system. Both the rate of replication and the rate of mutation outpace the immune system's ability to respond. This means that, while the immune system is capable of mounting an effective response to a given type of HIV particle, enough new variants of the particle are produced to stay ahead of the immune system. If replication of the wild-type virus can be suppressed either before the immune system is substantially damaged or long enough to allow the immune system to recover, the vaccine of the present invention can be used to strengthen the immune system's ability to recognize the new variants of the virus, thereby providing a means of controlling viral replication in individuals that have already been infected.

Drug combinations that are effective to at least temporarily inhibit HIV replication are known. The inventors have shown that drug combinations including hydroxyurea, one or more reverse transcriptase inhibitors and, optionally, one or more protease inhibitors are particularly effective, and, for some patients, allow the possibility of stopping drug treatment for extended periods of time. See U.S. Ser. No. 09/056,691, filed Apr. 8, 1998 “Method of Inhibiting HIV by Combined Use of Hydroxyurea, a Nucleoside Analog, and a Protease Inhibitor, U.S. Ser. No. 09/048,753 filed Mar. 26, 1998 Method of Inhibiting HIV using Hydroxyurea and Reverse Transcriptase Inhibitor in vitro and U.S. Ser. No. 09/048,756, filed Mar. 26, 1998, Method of Rendering a HIV Population Replication Incompetent in vivo, all of which are incorporated herein by reference as if set forth in full. The present invention includes the treatment of a patient with active HIV infection with an appropriate drug combination until the viral load in the blood has been effectively suppressed, that is, has reached a suitably low level, less than about 50,000 copies per ml, preferably less than 10,000 copies per ml, more preferably less than 200-500 copies per ml. The patient is then vaccinated using the present invention while the drug combination suppresses replication of the wild-type virus.

Drugs Suitable for Use in the Present Invention

Current antiretroviral drug regimens typically rely on one or more reverse transcriptase inhibitors, protease inhibitors, and a variety of other drugs including immune system treatments and a variety of unique agents, and may include 2-4 (or more) compounds, administered together. Highly active antiretroviral therapy a name commonly used in the field of HIV infection to mean combinations of three or more drugs, including at least one reverse transcriptase inhibitor and one protease inhibitor, or any combination of the drugs described below might be used according to the present invention for treatment of HIV infection. Hydroxyurea-containing combinations are preferred.

Hydroxyurea is one of many inhibitors of ribonucleotide reductase, an enzyme known for catalyzing the reduction of ribonucleoside diphosphates to their deoxyribonucleoside counterparts for DNA synthesis. Hydroxyurea inhibits viral replication, and also acts to down-modulate the immune system. Another material that inhibits viral replication and down-modulates the immune system is cyclosporine, a cyclophilin inhibitor. Other ribonucleotide reductase inhibitors include guanazole, 3,4-dihydroxybenzo-hydroxamic acid, N,3,4,5-tetrahydroxybenzimidamide HCl, 3,4-dihydroxybenzamidoxime HCI, 5-hydroxy-2-formylpyridine thiosemicarbazones, and n-(N)-heterocyclic carboxaldehyde thiosemicarbazones, 4-methyl-5-amino-1-formylisoquinoline thiosemicarbazone, N-hydroxy-N′-amino-guanidine (HAG) derivatives, 5-methyl-4-aminoisoquinoline thiosemicarbazone, diaziquone, doxorubicin, 2,3-dihydroxylbenzoyl-dipeptides and 3,4-dihydroxylbenzoyl-dipeptides, iron-complexed 2-acetylpyridine 5-[(2-chloroanilino)-thiocarbonyl]-thiocarbonohydrazone (348U87), iron-complexed 2-acetylpyridine-5-[(dimethylamino)thiocarbonyl]-thiocarbonohydrazone (A1110U), 2′-deoxy-2′-methylenecytidine 5′-diphosphate (MdCDP) and 2′-deoxy-2′, 2′-difluorocytidine 5′-diphospahte (dFdCDP), 2-chloro-9-(2-deoxy-2-fluoro-β-D-arabinofuranosyl)-adenosine (Cl-F-ara-A), diethyldithiocarbamate (DDC), 2,2′-bipyridyl-6-carbothioamide, phosphonylmethyl ethers of acyclic nucleoside analogs, [eg. diphosphates of N-(S)-(3-hydroxy-2-phosphonylmethoxypropyl and N-2-phosphonylmethoxyethyl) derivatives of purine and pyrimidine bases], nitrosourea compounds, acylclonucleoside hydroxamic acids (e.g., N-hydroxy-n-(2-hydroxyethoxy)-1(2H)pyrimidineacetamides 1-3, and 2-acetylpyridine 4-(2-morpholinoethyl)thio-semicarbazone (A723U)).

In human chemotherapy, hydroxyurea is currently administered using two basic schedules: (a) a continuous daily oral dose of 20-40 mg per kg per day, or (b) an intermittent dose of 80 mg per kg per every third day. Either schedule could be used in the treatment of viral infections. Lower dosages of hydroxyurea may also be effective in treating HIV infections. The presently preferred dosage range for use of hydroxyurea in treating HIV infections is 800-1500 mg per day, which can be divided over a 24 hour period, for example as 300-500 mg three times a day (TID), 500 mg twice a day (BID), or 1,000 mg once a day (QD), assuming an adult weighing about 70 kg. When the patient's weight is over 60 kg, 400 mg TID is preferred, for those under 60 kg, 300 mg TID is preferred.

Reverse transcriptase inhibitors figure prominently in current HIV treatments. Examples include nucleoside analogs, such as the 2′,3′-dideoxyinosine (ddI)(available as Videx® from Bristol Myers-Squibb). Nucleoside analogs are a class of compounds known to inhibit HIV, and ddI is one of a handful of agents that have received formal approval in the United States for clinical use in the treatment of AIDS. Like zidovudine (3′-azido-2′,3′-dideoxythymidine or azidothymidine [AZT] available from Glaxo Wellcome), zalcitabine (2′,3′-dideoxycytidine [ddC] available as Hivid® from Hoffman-La Roche), lamivudine 2′-deoxy-3′-thiacytidine [3TC](Epivir® available from Glaxo Wellcome), Iodenosine (F-ddA available from US Biosciences and stavudine (2′,3′-didehydro-2′,3′-dideoxythimidine [D4T] available as Zerit® from Bristol Myers-Squibb), ddI belongs to the class of compounds known as 2′,3′-dideoxynucleoside analogs, which, with some exceptions such as 2′,3′-dideoxyuridine [DDU], are known to inhibit HIV replication, but have not been reported to clear any individual of the virus. Other nucleoside reverse transcriptase inhibitors include adefovir (Preveon® an adenine nucleotide analog from Gilead Sciences), abacavir (1592U89 available from Glaxo Wellcome), lubocavir (a guanosine analog available from Bristol Meyers-Squibb), and PMPA, available from Gilead Pharmaceuticals. New nucleosides include FTC (Emtricitabine), DAPD, also known as DXG, F-ddA (Lodenosine, a fluorinated purine nucleoside RTI, and dOTC (BCH-10562). Non-nucleoside reverse transcription inhibitors include nevirapine (Viramune™ available from Boehringer Ingelheim Pharmaceuticals, Inc.), delaviridine (Rescriptor® available from Pharmacia & Upjohn) and efavirenz (available as Sustiva®, from DuPont Merck)

Currently, antiviral therapy requires doses of ddI at 200 mg per day BID for an adult human, or in the alternative 400 mg once a day (QD). Similar dosages may be used in the present invention. However, use of combinations of drugs may increase the effectiveness of these nucleoside phosphate analogs so that they can be used at lower dosages or less frequently. In combination with hydroxyurea, the presently preferred range for ddI is 100-300 mg twice a day (BID) or 400 mg once a day (QD), assuming an adult weighing 70 kg. When d4T is used with either hydroxyurea or a combination of hydroxyurea and ddI, the preferred range is 40 mg BID.

Of the potential protease inhibitors for use against HIV, compounds such as hydroxyethylamine derivatives, hydroxyethylene derivatives, (hydroxyethyl)urea derivatives, norstantine derivatives, symmetric dihydroxyethylene derivatives, and other dihydroxyethylene derivatives have been suggested, along with protease inhibitors containing the dihydroxyethylene transition state isostere and its derivatives having various novel and high-affinity ligands at the P2 position, including 3-tetrahydrofuran and pyran urethanes, cyclic sulfolanes and tetrahydrofuranylglucines, as well as the P3 position, including pyrazine arnides. In addition, constrained “reduced amide”-type inhibitors have been constructed in which three amino acid residues of the polypeptide chain were locked into a g-turn conformation and designated g-turn mimetics. Other alternatives include penicillin-derived compounds and non-peptide cyclic ureas. βuitable protease inhibitors include Indinavir sulfate, (available as CrixivanTM capsules from Merck & Co., Inc, West Point, Pa.), saquinavir (Invirase® and Fortovase® available from Hoffman-LaRoche), ritonavir (Norvir® available from Abott Laboratories) ABT-378 (available from Abott Laboratories), Nelfinavir (Viracept®), and GW141 (available from Glaxo Wellcome/Vertex) Tipranavir available from Pharmacia & Upjohn, PD 178390 available from Parke-Davis, BMS-23632 available from Bristol-Myers Squibb, DMP-450 available from Triangle, and JE 2147 available from Agouron. New protease inhibitors include ABT-378 (Abbott laboratories), L-756423, DMP-450 and AG1776.

In addition to reverse transcriptase inhibitors and protease inhibitors, the present invention may utilize integrase inhibitors such as AR177 (Zintenvirt available from Aronex); fusion inhibitors such as pentafuside, (T-20) and cytokine inhibitors (available from Chiron), chemokine inhibitors, and antisense oligonucleotides such as GPI-2A available from Novopharm Biotech, ISIS-13312 available from Isis, and GEM-132 and GEM-92 available from Hybridon. Other compounds which might be used include mycophenolic acid (MPA, available from Cellcept) and PRO 52 (CD4-Ig2), a fusion protein comprising a human immune globulin in which parts of the heavy and light chains have been replaced with domains from the human CD4+ cell.

Suitable human dosages for these compounds can vary widely. However, such dosages can readily be determined by those of skill in the art. For example, dosages to adult humans of from about 0.1 mg to about 1 g or even 10 g are contemplated.

The following Examples are presented for the purpose of illustrating the practice of the present invention. They do not limit the invention, or the claims which follow.

### EXAMPLES

1. Expression of Plasmid DNA Encoding a Replication Defective HIV in Cultured DC

There are several sources of DC. DC can be isolated from bone marrow CD34+ hematopoietic progenitor cells. Bone marrow mononuclear cells will be separated by Ficoll-Hypaque gradient centrifugation. These cells will be positively selected with human CD34 antibodies conjugated magnetic beads (Dynal Detachabeads) and CD34+ cells will be displaced from magnetic beads using high affinity polyclonal antibody against CD34 monoclonal antibody. These cells can differentiate to DC when they are cultured with stem cell factor, GM-CSF and TNF-alpha [Canque, B., M. Rosenzwajg, et al. (1996). “The effect of in vitro human immunodeficiency virus infection on dendritic-cell differentiation and function.” Blood 88(11): 4215-28.]

Monocyte-derived DC were generated from peripheral blood mononuclear cells in the presence of GM-CSF and IL-4. [Bender, A., M. Sapp, et al. (1996). “Improved methods for the generation of dendritic cells from nonproliferating progenitors in human blood.” J Immunol Methods 196(2): 121-35.] On day 4, cells were transfected with lipofectamine complexed with plasmid DNA encoding HIV-1/LWint- (an integration and replication defective HIV described in U.S. Ser. No. 08/803,484). Lipofectamine, a commercially available cationic liposome useful as a transfection reagent (available from Gibco BRL Life Technology, PM. Gaithersburg, Md., US)

48 hours later cells were washed and analyzed. The purity of the DC, characterized by Fluorescence Activated Cell Sorter (FACS) measuring surface markers (FACS) was 90.6%. DC cell types were found to be CD3-, CD19-, CD56-, CD14-and HLA DR+ using FACS analysis. The expression of HIV-1 Gag and Env and Tat proteins was also measured by FACS in permeabilized cells. The level of non-specific binding of isotope control Ig was the same in the control transduced and specific plasmid transduced. We found in three independent experiments that 25-37% of HIV-1/LWint-transduced DC expressed Env, Gag and Tat proteins. That is, 25-37% of the cells in the transduced samples expressed HIV proteins. Transduced and control cell samples were also double-stained with p24 and B7-2 antibodies to demonstrate that DC and not macrophages were expressing the antigen. These results were surprisingly good, because using the same methods with another plasmid DNA (CMV-driven hemagglutinin of influenza virus gene) only 5-8% of the transfected cells expressed proteins. These results demonstrated that defective HIV can be efficiently expressed by transduced DC.

2. DNA Encoding Replication Defective Viruses are More Efficient Antigens Than DNA Encoding One or More Proteins

In an independent experiment we compared the expression of two different HIV plasmids in DC: HIV-1/LWint- and LTR-tat. Both constructs are driven by the same promoter: HIV-1-LTR and the expression of both constructs depends on the transactivation of Tat. Transfection was performed as described in example 1 and 48 hours later expression of the Tat protein was analyzed by FACS. We found that 32% of HIV1/LWint- plasmid transfected DC expressed Tat protein. In contrast only 10% of the LTR-tat transfected DC expressed the same Tat protein. This result was suprising, because in this comparative study we were expected the same efficiency with the two different constructs. Replication defective viruses definitly have the capability to form viral particle, which can be released from the cell. Since antigen presentation depends on gene expression in DC, this experiment clearly demonstrate that DNA encoding defective viruses are more efficient antigens than DNA encoding one or more proteins.

3. Transduced, Cultured DC Activate Naive CTL in vitro

After transduction, DC were cultured with autologous T-cells at a ratio of 1:10. After 7 days these T-cells were used as an effector to lyse (kill) a monocyte/macrophage cell population from the same donor that had been pulsed with p55, an HIV protein conveniently used as a label. This CTL activity was measured using Cr-relase assay. As FIG. 5 demonstrates, CTL induced by transduced DC lysed the target cells specifically and effectively. Because generation of CTL in vitro is more difficult than in vivo, these experiments show that cells which have been subjected to genetic immunization can activate naive CTL so that they will effectively lyse in vivo infected cells. Furthermore, the experiment farther demonstrates that DNA encoding a defective virus not only express efficiently HIV genes but also can generate an effective immune response.

4. Ex Vivo Genetic Immunization with Transduced DC

To prove the in vivo efficacy of genetic immunization in monkeys, dendritic cells were generated from 40 ml peripheral blood of pigtail macaques. Cells were transfected with LW/int- plasmid using polyethylenimine as described in Example 5. The transfected DC were washed and injected into juvenile pigtail macaques 36-48 hours after transfection. One part of the transfected DC was injected subcutaneously and one part was injected intravenously. After 4 weeks and only one immunization attempt, one monkey already showed CTL response (FIG. 6), which suggests that the in vitro result can be reproduced in vivo in animals.

5. Polyethyleneimine-mediated Gene Transfer into Cultured Dendritic Cells

Dendritic cells were transduced with plasmid encoding HIV-1 /LWint- as in Example 1, except that polyethyleneimine (PEI kindly supplied by Dr. Behr) was used instead of lipofectamine. The cells were tested as in Example 1, and more than 60% of the dendritic cells transduced using polyethyleneimine expressed HIV-1 proteins in contrast to the 25-37% of cells transduced using lipofectamine. Since up to date lipofection was the best gene transfer method to introduce plasmid DNA into DC, this experiment demonstrates that PEI is the most efficient non viral gene delivery system to transfer genes into DC. However, both PEI and lipofectamine exhibited significant toxicity to dendritic cells, as measured by tripan blue staining.

6. Specifically Targeting DC via Mannose Receptor

Immature DC were generated as described above in Example 1 and transfected with DNA encoding a green fluorescent protein (GFP). We used this DNA as a marker for gene expression, because cells expressing the green fluorescent protein light up green after the fluorescent stimulation. Therefore, transfected cells can be visualized by fluorescent microscopy and flow cytometry (FACS).

PEI modified with different sugars was chosen to target the mannose receptor on the surface of dendritic cells because the mannose receptor recognizes all the patterns of sugars on the surface of bacteria, parasites, yeast and fungi. DNA was complexed with PEI and with different sugar-bearing polyethylenimines (available on a custom order basis from Dr. Jean-Paul Behr, Laboratoire de Chimie Genetique, Faculte de Pharmacie, CNRS-UMR 7514 74 route du Rhin 67401 Illkirch, France). 2 microgram DNA was incubated with different derivates of PEI in 150 mM NaCl (10:1 N:P ratio) at room temperature for about 5 minutes. Than DC were transduced with the complexes for 6 hours washed, and green fluorescent cells were analyzed after 48 hours. We found that the most effective PEI-sugar modification is the PEI-mannose (Table 1).

TABLE 1
Different Complexes for in vitro Transduction of Dendritic cells

| | % of cells expressing green |
|---|---|
| Experiment | fluorescent protein |
| 1. Control | 4 |
| 3. PEI-mannose-DNA | 43 |
| 4. PEI-galactose-DNA | 23 |
| 5. PEI-glucose-DNA | 19 |

7. In Vitro PEI-Mannose-mediated Gene Transfer into Cultured Dendritic Cells

The mannose-bearing polyethylenimine (PEI-man) is an isothiocyanantophenyl phenyl mannose derivative, coupled to PEI 25 kDa, yielding a ligand (or, mannose residue of low affinity for the mannose receptor, 1 mM). It has been previously demonstrated that entry via the asialoglycoprotein receptor (used by PEI) requires the complex to be charged. That is, more PEI than DNA must be used. When the complex is neutralized, that is, the PEI is neutralized by the DNA, the complex cannot enter via the asialoglycoprotein receptor. [Zanta M A; Boussif O; Adib A; Behr J P. Bioconjug Chem 1997 November-December;8(6):839-44]: These investigators developed a thought to favoring vivo gene delivery to the liver: 1) electrostatically neutral particles which avoid nonspecific binding to other cells, and 2) to avoid asialoglycoprotein receptor-mediated endocytosis. This system was based on a 5% galactose-bearing polyethylenimine (PEI-gal) polymer which is condensed with plasmid DNA to neutrality.

We found that, with PEI-man-DNA complexes, less DNA is required to neutralize PEI-man compared to PEI. Gel electrophoresis experiments using different N:P ratio of PEI-DNA complexes demonstrated that 5:1 (N:P) of PEI-man:DNA complex has neutral charge, in contrast to 3:1 (N:P) PEI-DNA complex. {The neutralization of PEI with the DNA depends on the N(nitrogen):P(phosphate) ratio; one microgram DNA=3×109 molar P and 1 mM PEI=109 molar N/microliter. This means for example that 10:1 ratio is the mixture of 3 microliter 10 mM PEI and 1 microgram DNA.}

Human DC were isolated as described above. Purity of DC, characterized by FACS, was over 99%. By measuring cell viability with tripan-blue staining, we found that PEI-man was much less toxic than PEI. We also found that both PEI and PEI-man are able to introduce DNA into DC, however PEI-mannose is more efficient. At 5:1 (N:P) ratio PEI-man-DNA complex is neutral, therefore the complex is only able to enter into DC via the mannose receptor. Under these conditions, 30% of the DC were expressing the green fluorescent protein. In contrast, 5:1 (N:P) ratio PEI-DNA complex is charged and the complex enters via asialoglycoprotein receptor. Under this condition, 14% of the DC expressed the green fluorescent protein (FIG. 4).

8. In Vivo Gene Delivery to Skin Langerhans Cells

In the skin, the only cells which can endocytose mannosylated ligands are the Langerhans cells. Therefore, the main question was whether the PEI-(man) complex can penetrate into the skin and transfect Langerhans cells in vivo. PEI-(man) was complexed with plasmid DNA encoding a green fluorescent protein (GFP). Experiments with different gene delivery complexes were performed as described in FIG. 3. The complex contained 50 microgram DNA and 8.25 microliter 100 mM PEI-man in a 5-10% glucose solution (optimum=8%). BALB/c mice were anaesthetized, and the backs of the mice were shaved. 0.1 ml of the formulation with the complexes was applied on the skin for one hour or subcutaneously as indicated in the Table 2. Mice were sacrificed 6 hours after immunization and skin samples were placed in DMEM media supplemented with 10% fetal calf serum and antibiotics. Under these conditions cells, including Langerhans cells migrate out from the skin. One day later the migrating cells were collected and analyzed by flow cytometry (FACS), because this analysis can recognize cells expressing the green fluorescent protein. In our analysis only the large and dense cell population was analyzed, because both dendritic cells and Langerhans cells are known to be large, dense cells.

TABLE 2
In vivo transduction of skin Langerhans cells

| | % of cells expressing green |
|---|---|
| Experiment | fluorescent protein |
| 1. Control | 0.84 |
| 2. Subcutaneous PEI-DNA | 0.20 |
| 3. Subcutaneous DNA | 1.74 |
| 4. Transcutaneous PEI-DNA | 6.52 |
| 5. Transcutaneous DNA | 29.10 |
| 6. Transcutaneous PEI-man-DNA | 22.99 |

These experiments demonstrate that 1) transcutaneous gene delivery results in more efficient gene transfer into Langerhans cells than subcutaneous delivery (compare experiment 2 & 4 or 3 & 5). This is important, because one of the best present vaccination technologies uses subcutaneous injection. 2) Entry via mannose receptors is more efficient to transfect in vivo Langerhans cells than entry via asialoglycoprotein receptor (compare experiment 4 & 6). These in vivo experiments confirm our in vitro experiment (see FIG. 4). Therefore, the sugar modified gene delivery system is preferred to transduce antigen presenting cells.

9. Transduced Langerhans Cells Migrate to the Lymph Nodes

BALB/c mice were prepared as in Example 8, and 0.1 ml samples of the PEI-man-DNA complex were applied on the skin for one hour. 2 days later the animals were sacrificed and lymph nodes (LN) were removed. Auxiliary LN were investigated because they are the draining LN of the back, and migrating Langerhans cells might be found there. The LN were frozen, sliced and examined under fluorescent microscope. The LN of experimental mice were compared with the LN of a control mouse. We were able to detect about 15 green fluorescent cells in the samples from the experimental LN and none in the control LN. These results demonstrate that the complex entered into cells located in the skin, and the cells were able to migrate into the LN and express the green fluorescent protein. The morphology of these green cells resembles DC morphology: these are big cells and the localization of the green fluorescence shows a “bumpy” pattern, which is characteristic to DC. (Other cells, e.g. 293 cells show a diffused green pattern in the cytoplasm.) In addition, the only cell type is able to pick up antigens and migrate to the LN is the Langerhans cell. These cells are the only cells in the skin to bear the mannose receptor in order to take up the complex and after activation is known that they are migrating in the draining LN.

These experiments show that PEI-(Man)-DNA complexes are able to penetrate in the skin, and deliver the DNA into Langerhans cells. The Langerhans cells were activated and migrated into the draining LN and expressed genes encoded by the DNA construct in the LN. It is known that cultured DC reinjected to the body migrate in the LN and generate efficient immune response. This invention demonstrates that in vitro isolation of DC is not required to transfer genes into Langerhans cells, or for gene expression in the lymphoid organs. We have also demonstrated that expression of a replication defective virus in DC results in efficient induction of a CTL response in vitro and in vivo (see above). Therefore, we have shown that transcutaneous gene delivery with complexes (like PEI-man-DNA) can be utilized to generate immune responses against proteins encoded in the DNA.

10. Sugar-DNA Complexes to Transduce Skin Langerhans Cells

Experimental results depicted in Table 2 provided evidence that a sugar-DNA complex, in the absence of PEI-man, can transduce Langerhans cells in vivo. Sugar complexed DNA in the absence of PEI is more efficient for use in both subcutaneous and transcutaneous methods than DNA complexed with PEI (see Table 2, experiments 3 & 5). This is a very surprising result. It shows that sugars (e.g. 8% glucose in these experiments) can also complex DNA and deliver the DNA to the Langerhans cells via the mannose receptor. Importantly, the most efficient gene delivery in vivo to the Langerhans cells was the sugar complexed DNA used in the transcutaneous way.

We expect that immunization with the sugar-DNA complex would also result in migration of the Langerhans cells to the draining lymph node. The reason is that the same mechanism is utilized for entry to Langerhans cells: mannose-receptor mediated uptake. The advantage of using sugars as adjuvants in the presently used vaccination technologies is that higher percentages of Langerhans cells will be involved in generation of immune response. This is expected to significantly increase the efficacy of present vaccination strategies. For example, mixing vaccines with sugar for subcutaneous, intradermal and intramuscular injections of DNA and protein antigens.

11. Implications of Transcutaneous Immunization

This technology would revolutionize the immunization methods because no needles are required. The described transcutaneous immunization is a very simple technology which could be used for inexpensive vaccination in both the developed and developing world.

The simplicity of the methodology and the fact that the antigen presenting cells are transduced efficiently enables us to use any DNA sequence able to generate an immunogenic protein. Thereby, the broadest spectrum of diseases can become target of immunization, e.g. infectious diseases and cancer. Further, the only practical way to eradicate certain infectious diseases, like HIV infection, hepatitis, malaria, is a simple inexpensive vaccination. Finally, vaccines without needle sticks will be especially welcome to the parents of small children.

Use of the present invention potentially allows the development of safer vaccines. The few negative reactions to classical vaccines are sometimes due to an allergic reaction to byproducts of the vaccine manufacturing process. Where such additional materials (egg albumin, for example) can be eliminated, the rate of negative reactions can be concomitantly reduced. Another attractive aspect of this invention is that it can be used for the treatment of diseases with or without drug treatments such as combination antiretroviral therapy or chemotherapy.

12. Raising an Immune Response—Two Animal Models

Here we describe an alternative, non-invasive, transcutaneous DNA immunization technology to transduce a large number of lymph node DC with a plasmid DNA and induce potent T cell mediated immune responses.

Methods

Synthesis of PEI-mannose (PEIm). Linear polyethylenimine of 22 kDa was synthesized from the commercially available polyethyloxazolidine (Aldrich). It was glycosylated by reductive amination in the presence of sodium cyanoborohydride, as adapted from Zanta, M. A., Boussif, O., Adib, A. & Behr, J. P. In vitro gene delivery to hepatocytes with galactosylated polyethylenimine. Bioconjug Chem 8, 839-844 (1997). For the coupling reaction, Man α4 Man (100 μmols, 35.2 mg, Sigma, St Quentin Fallavier, France) and sodium cyanoborohydride (500 μmol, 31.4 mg, Aldrich, St Quentin Fallavier, France) were added to linear 22 KDa PEI 2 mmol of monomer, 2 ml of 1 M PEI pH 7.0 stock solution) in 10 mnl 0.2 M borate buffer, pH 8.4 (sterilized by filtration through a 0.22μ filter), and left for 48-72 h at room temperature with stirring. The mixture was transferred with a sterile Pasteur capillary pipette to a sterile dialysis membrane (cellulose ester with Mw cutoff 3,500 Da). Low molecular weight compounds (unreacted Manα4Man and boron salts) were removed by dialysis against 1 L of ultra-pure H₂O at room temperature for 1 day (with 3 water change). The polymer-containing solution was transferred into a 20 mL sterile syringe, filtered through a 0.22μ membrane and aliquoted (0.5 mL) in sterile storage tubes.

The average number of glycosyl residues per PEI molecule was determined by using the resorcinol/sulfuric acid micromethod (Monsigny, M., Petit, C. & Roche, A. C. Colorimetric determination of neutral sugars by a resorcinol sulfuric acid micromethod. Anal Biochem 175, 525-530 (1988). The level of substitution was expressed as a percentage of pentamannosyl residues per amino group of PEI and found to be 2.3±0.1%. The concentration of PEI was determined by agarose gel electrophoresis in the presence of plasmid DNA. DNA was entirely retained in the well for a PEI amine/DNA phosphate (N/P) ratio above 2 (data not shown).

Zeta potential and particle size measurements. DNA (0.05 mg) and various amounts of PEIm were diluted separately in 0.5 mL of 8% glucose. After 10 min, the PEIm was added to the DNA; the resulting solution was homogenized and left for 15 min at room temperature. Particle size was determined by light scattering using a Zetasizer (Malvern Instrument, Orsay, France) with the following specifications: 3 measurements per sample; medium viscosity, 1.014 cP; refractive index (RI) medium, 1.34; temperature, 20° C. Particle zeta potential was measured with the same apparatus and with the following specifications: sampling time, 30 s; 3 measurements per sample; viscosity, 1.014 cP; dielectric constant, 80.37; temperature, 20° C.; cell drive voltage, 250 V; beam mode F(Ka)=1.50 (Smoluchowsky).

Plasmid DNA. A plasmid, known to express green fluorescent protein and neomycin phosphotranferase, pGFP, was utilized in most of the experiments (Clonetech, Palo Alto Calif.). A plasmid expressing the HIV-1 gag protein, Gag-delta8.2, (Naldini, L. et al. In vivo gene delivery and stable transduction of nondividing cells by a lentiviral vector. Science 272, 263-267 (1996)) was kindly provided by Inder Verma.

Construction of DermaVir_{SHIV}: pSHIV(int-) viral vector. The fall length but integration defective Simian Human Immunodeficiency Virus (SHIV) clone [pSHIV(Int-1)] to obtain DermaVir_{SHIV} was created by stepwise strategy starting with p-5′SHIV (clone KB9) and p-3′SHIV (clone 64/KB9) provided by Joseph Sodroski of Harvard University. These clones, derivatives of SHIV 89.6, are also available from the NIH AIDS Research and Reference Program. First a clone of p-5′SHIV with the deletion of the internal Bg12-Bg12 sites located in the pol gene was created and termed p-5′_{SHIV}(dBg). The internal Bg12-Bg12 fragment was mutated by PCR amplification of the fragment with primers that introduced mutations with stop codons and cloned into a separate vector and termed pBg08. The mutated fragment was isolated and inserted into p-5SHIV(dBg) to obtain P-5′_{SHIV}(Int-1). The Xhol-Sphl viral fragment (˜6.5 Kb) from p-5′_{SHIV}(Int-1) and Sphl-Notl viral fragment (˜4.0 Kb) from p-3′SHIV clones were isolated and cloned into a pBluescript (Stragene, Inc.) vector backbone to obtain p_{SHIV}(Int-1) clone. The sequence of the junctions and of the integrase gene region of this clone was checked. It contained small deletions, frame shift and three separated stop codons in the integrasegene open reading frame. It also contained stop codons in the other reading frame in this region. SIVmac₂₃₉ sequence 1: (nt 4696) 5′-A GAT CTA GGG ACT TGG CAA ATG GAT TGT ACC CAT-3′ (nt 4729). p_{SHIV}(Int-1) sequence 2: 5′-A GAT CTA TGA-----TAG----A TAG CT TAG---CC CAT-3′.

Animals. All animal experiments were performed under protocols approved by the Animal Care and Use Committee. Unless otherwise indicated, 4-6-week old female BalbC mice were used. The mice were anesthetized using methoxyflurane.

Non-human primate studies were performed with animals assigned to an approved Animal Care and Use protocol. For the transcutaneous gene transfer studies, the animals were initially sedated with Ketamine Xylazine and placed on a circulating water heating pad. An endotracheral tube was placed and the animal was maintained on 1.5% isofluorane anesthesia for the duration of the experiment.

Transcutanoeus gene transfer. The backs of the mice or the inner thighs of the monkeys were shaved with an electric razor, washed with 70% EtOH, and dried. Then the skin was lightly rubbed when indicated using a scrub sponge (3M Scotch-Brite Heavy Duty Scrub Sponge) five to fifty times. For monkeys, 0.2 mL PEIm/DNA complex was prepared shortly before application using 0.025 mg DNA and 0.0136 mL 50 mM PEIm (N/P=9) in 8% glucose-water solution. A sample of 0.2 mL of the complex was applied on a ca. 20 cm² area of the monkey's inner thigh skin with a pipette. After 40 min of contact time under general anesthesia, a non-absorbent pad was placed over the site and a bandage applied to extend the total contact time until the animal recovered. The animal was returned to the cage once it had fully recovered, and was monitored for any reaction to the procedure.

For mice, the same complex was prepared in 0.1 mL 5% glucose-water solution and applied on about 4 cm² area on the back.

Detection of genetically modified cells in the skin. The PEIm/DNA complex (plasmid DNA: pGFP) was applied onto the surface of the skin or injected subcutaneously in mice. For control samples, the skin was prepared the same way, but treated with only 8% glucose solution. The animals were sacrificed 6 hours after treatment, the shaved skin from the back was removed, scraped with a sterile blade in the direction of the most prevalent skin veins and placed in culture media (DMEM with 10% FCS and antibiotics). The skin sections were removed from the culture 24 hours later and the cells migrating out from the explant were centrifuged at 1500 rpm for 5 min, washed two times with PBS and analyzed by flow cytometer (Becton Dickinson).

In situ hybridization and immunohistochemical staining. In situ hybridization was conducted using the basic principles (Fox, C. H. & Cottler-Fox, M. In situ hybridization in HIV research. J Microscop Tech Res 25, 78-84 (1993)) and protocol (Fox, C. H. & Cottler-Fox, M. in Current Protocols in Immunology (eds. Coligan, J., Kruisbeek, A., Margulies, D., Shevach, E. & Strober, W.) (Wiley, New York, 1993)) that have also been used as a standard protocol for a number of different targets. Riboprobes are ³³P labeled and have been determined to detect 20-30 copies per cell of HIV gag RNA, although in the case of Neo probes the sensitivity is somewhat less. The slides were exposed for five days before development and examination by dark-field microscopy. Immunohistochemistry was performed using protocols recommended by the supplier of the primary antibodies.

Composition and preparation of DermaVir_{SHIV}

| Solution 1 | Solution 2 |
|---|---|
| 0.054 mL of 25 mM PEIm | 0.100 mL of p_{SHIV}(Int-) DNA (1 mg/ml) |
| 0.346 mL of 10% Glucose | 0.300 mL of 10% Glucose |

Solutions must be separately vortexed vigorously. Solution 1 was added to solution 2 in a 1.8 ml sterile Eppendorf tube and vigorously pipetted up and down 20 times.

Quantitative determination of SIV-specific T cell responses (VIR assay). The VIR assay was performed as previously described (Lori, F. et al. Control of SIV rebound through structured treatment interruptions during early infection. Science 290, 1591-1593. (2000)). PBMC were plated in round-bottom 96-microtiter plates (Costar, NY) at 0.5 million cells/well in 0.1 mL complete RPMI 1640 medium containing 2 μg Zinc-finger-inactivated SIV or HIV (kindly provided by Jeff Lifson, NCI, Frederick Md) or mock antigen (2 μg lysozyme) and 50 IU/ml rhIL-2 (gift from Hoffinan La Roche). Cells were cultured for 15 hours and treated with Brefeldin A (Sigma, USA) at 10 μg/mL for an additional 3 hours. Cells were collected and aliquoted into 0.5 million cells per test tube. After washing once with 2 mL PBS containing 1% BSA, cells were suspended in 0.1 mL PBS/1% BSA and stained with CD8 and CD3 fluorescent antibodies for 15 min at room temperature. After washing, cells were fixed with 2% paraformaldehyde, pH 7.4 for 10 min and washed with PBS/1% BSA, then permeabilized with 0.1 mL 0.1% saponin in PBS/1% BSA for 5 min and stained with anti-interferon-gamma (anti-IFN-γ) antibody for 15 min at room temperature. After intracellular stained cells were washed twice with 1 ml PBS, re-suspended in a 0.5 ml 1% paraformaldehyde PBS buffer. Samples were analyzed on FACS (EPICS XL-MCL, Coulter). A total of 50,000 events were acquired. Gated CD3⁺ CD8⁺ cells were examined for staining with interferon gamma (IFN-γ). The numbers of IFN-γ producing CD8⁺ T cells were calculated per million of total CD8⁺ T cells; similar to the ELISPOT convention that uses the number of IFN-γ producing cells per million cells. Calculation of CD8VIR=number of IFN-γ⁺ cells (specific antigen activation)-number of IFN-γ⁺ cells (no antigen activation).

Results

We hypothesized that a small size DNA-containing complex applied on the surface of skin could reach the LC because these cells are located right below the upper layer of the stratum comeum. If a complex mimics a bacterial antigen, it could trigger LC to take up the complex and migrate into the lymph nodes (Sallusto, F., Cella, M., Danieli, C. & Lanzavecchia, A. Dendritic cells use macropinocytosis and the mannose receptor to concentrate macromolecules in the major histocompatibility complex class II compartment: downregulation by cytokines and bacterial products. J Exp Med 182, 389-400 (1995); Engering, A. J. et al. The mannose receptor functions as a high capacity and broad specificity antigen receptor in human dendritic cells. Eur J Immunol 27, 2417-2425 (1997); Engering, A. J. et al. Mannose receptor mediated antigen uptake and presentation in human dendritic cells. Adv Exp Med Biol 417, 183-187 (1997)). Since bacteria enter via the mannose receptors into LC, we wanted to explore the same mechanism of entry. The mannose receptor is exclusively expressed on the surface of terminally differentiated macrophages and LC (Takahashi, K., Donovan, M. J., Rogers, R. A. & Ezekowitz, R. A. Distribution of murine mannose receptor expression from early embryogenesis through to adulthood. Cell Tissue Res 292, 311-323 (1998)); therefore, it represents a good target for in vivo gene transfer to the LC of the epidermis. It has been previously demonstrated that polyethylenimine-mannose (PEIm) and DNA complexes can specifically target mannose receptors on immature DC (Diebold, S. S., Kursa, M., Wagner, E., Cotten, M. & Zenke, M. Mannose polyethylenimine conjugates for targeted DNA delivery into dendritic cells. J Biol Chem 274, 19087-19094 (1999)). We postulated that since PEIm/DNA complexes enter into the endosomes of the LC by way of the mannose receptor, PEIm would buffer the endosome, thereby protecting the DNA from lysis, and also delivering the DNA into the nucleus of the cells where gene expression occurs. LC would then be triggered to migrate and express the gene in the draining lymph as DC. These DC can then prime naive T cells and induce T cell-mediated immune responses.

Formulation and characterization of the DNA for transcutaneous gene transfer. We have demonstrated that plasmid DNA complexed with PEI can transduce monocyte-derived immature DC in vitro and these DC could induce primary T cell responses (Lisziewicz, J. et al. Induction of Potent HIV-1-specific T Cell Restricted Immunity by Genetically-modified Dendritic Cells. J Virol (submitted) (2001)). Since LC are similar to immature DC we hypothesized that simply applying these complexes to the surface of the skin could transduce LC in the skin. To achieve this we had to minimize the size and optimize the stability of the PEIm/DNA complexes. We performed particle size measurements by laser light scattering in order to obtain information about the formation and behavior of PEIm/DNA complexes. Previous work with PEI/DNA complexes has suggested that complexes made in low salt solutions remained small and showed no tendency to aggregate (Erbacher, P. et al. Transfection and physical properties of various saccharide, poly(ethylene glycol), and antibody-deprived polyethylenimines (PEI). J Gene Med 1, 210-222 (1999); Goula, D. et al. Size, diffusibility and transfection performance of linear PEI/DNA complexes in the mouse central nervous system. Gene Ther 5, 712-717 (1998)). Therefore, we formulated the PEIn/DNA complexes without salt, in glucose solution. Our measurements showed that the size of the DNA complexes in glucose was between 80 and 100 nm (Table 3).

TABLE 3
Size and zeta potential of the PEIm-DNA complexes
in 8% glucose solution

| N/P ratio* | Mean Size (nm) | Zeta (mV) |
|---|---|---|
| 2 | 99.4 ± 5.6 | 21.1 ± 1.0 |
| 5 | 93.2 ± 4.6 | 51.7 ± 2.3 |
| 7 | 86.1 ± 2.3 | 56.3 ± 1.0 |
| 10 | 84.0 ± 3.9 | 57.2 ± 0.4 |

*(N/P is the molar PEI amine/DNA phosphate ratio)

At a seven molar equivalent PEIm amine per DNA phosphate ratio N/P=7), the mean size of the complexes was 410 nm (±41.8 nm) in 150 mM NaCl solution (routinely used for in vitro gene transfer), and 86.1 nm (±2.3 nm) in 8% isotonic glucose solution. The mean size of the latter complexes did not change after 24 h (83.1±4.7 nm) demonstrating the stability of PEIm/DNA complexes in solution at room temperature. These results indicated that small and stable particles could be obtained in glucose solution, by using an N/P ratio between 2 and 10.

Zeta potential controls the behavior of the particles in solution. Particles with higher zeta potential (more than 10 mV) are more stable and have a lower tendency to aggregate. We therefor determined the zeta potential of the complexes. We found that at an N/P ratio above 5, the zeta potential of the particles was over +50 mV (Table 3), thus ensuring colloidal stability of the complexes.

Based on this physico-chemical data, we prepared FITC (fluorescein isothiocyanate) labeled PElm/DNA-complexes at an N/P of 7 in 8% glucose and in 0.9% (150 mM) NaCl solution. Under fluorescence microscopy the PEI-DNA complexes in the glucose solution appeared small and their size did not change over time (up to 48 days). In contrast, the same complexes in a strong ionic solution (0.9% saline) rapidly formed aggregates (not shown).

Transcutaneous gene transfer to Langerhans cells. We first used a murine model to deliver a plasmid DNA encoding the Green Fluorescent Protein (pGFP) reporter gene for evaluating the in vivo transduction of LC. BalbC mice were shaved and the PEIm/DNA complex was applied on the surface of the skin. Three controls were included in this experiment: (i) mice were shaved and treated with glucose solution (negative control), (ii) mice were shaved and the complexes were injected subcutaneously (for comparison) and (iii) mice were shaved and fluorescein isothiocyanate-dextran (FITC-dextran) was injected subcutaneously (positive control). FITC-dextran injection was selected as an additional control, because it is a small diffusible molecule that is known to enter into LC and immature DC via the mannose receptor (Sallusto, F., Cella, M., Danieli, C. & Lanzavecchia, A. Dendritic cells use macropinocytosis and the mannose receptor to concentrate macromolecules in the major histocompatibility complex class II compartment: downregulation by cytokines and bacterial products. J Exp Med 182, 389-400 (1995). Six hours later the mice were sacrificed, the shaved part of the skin was removed and cultured as described previously, to allow the large LC and small T cells to emigrate from the explants into the medium (Larsen, C. P. et al. Migration and maturation of Langerhans cells in skin transplants and explants. J Exp Med 172, 1483-1493 (1990); Steinman, R., Hoffman, L. & Pope, M. Maturation and migration of cutaneous dendritic cells. J Invest Dermatol 105, 2S-7S (1995); Lukas, M. et al Human cutaneous dendritic cells migrate through dermal lymphatic vessels in a skin organ culture model. J Invest Dermatol 106, 1293-1299 (1996)).

The cells that migrated out from the skin into the culture media were harvested and the large cells containing the LC population were analyzed by flow cytometer (FIG. 7). In the control samples, about 0.3% of the cells migrating from the skin of the mice were green (FIG. 7a), defining the background value of the experiment. For mice treated with the PEIm/DNA complexes on the surface of the skin, 9.4% of the migrating cells expressed GFP (FIG. 7b). In contrast, after subcutaneous injection of PEIm/DNA complexes only 0.8% of the cells was green (FIG. 7c) suggesting that after injection the complexes could not diffuse to the epidermis and transduce LC. As expected from the positive control, subcutaneous injection of FITC-dextran resulted in 15.9% of green fluorescence cells (FIG. 7d) (Sallusto, F., Cella, M., Danieli, C. & Lanzavecchia, A. Dendritic cells use macropinocytosis and the mannose receptor to concentrate macromolecules in the major histocompatibility complex class II compartment: downregulation by cytokines and bacterial products. J Exp Med 182, 389-400 (1995). These results suggest that PEIm/DNA complexes applied on the surface of the skin transduced LC.

RNA expression in the lymph nodes after transcutaneous gene transfer. Next we examined whether in vivo PEIm/DNA transduced cells would migrate into the draining lymph node and express the gene encoded by the plasmid DNA. PEIm/DNA complexes were applied onto the surface of the skin of BalbC and C57BL/6 mice. A plasmid DNA encoding the Gag gene of HIV-1, designated as pGag (gift from Inder Verma, see Naldini, L. et al. In vivo gene delivery and stable transduction of nondividing cells by a lentiviral vector. Science 272, 263-267 (1996)), was used because HIV-1-gag mRNA could be detected with a quantitative RT-PCR assay (Bagnarelli, P. et al. Dynamics of molecular parameters of human immunodeficiency virus type 1 activity in vivo. J Virol 68, 2495-2502 (1994)). Control animals were similarly treated with PEIm/DNA complexes encoding GFP. The mice were sacrificed and the inguinal lymph nodes were removed 24 hours after gene transfer. These nodes were assayed for expression of HIV-1 Gag mRNA by RT-PCR (Table 4).

TABLE 4
Detection of HIV-1 Gag mRNA by RT-PCR in the lymph
nodes of mice after transcutaneous gene transfer.

| | | RT-PCR |
|---|---|---|
| Animal | Complex | (gag)copies/10⁶ cells |
| BalbC | PEIm/Gag | 32,800 |
| BalbC | PEIm/GFP | 3,516 |
| C57 | PEIm/Gag | 64,104 |
| C57 | PEIm/GFP | 3,420 |

Control RT-PCR in different species of untreated mice 4,536 (SD = 1610)

We found high levels of gag expression in the lymph nodes of mice treated with pGag containing PEIm/DNA complexes. Over 30,000 copies of gag mRNA per 10⁶ cells were found in the lymph node of either mice species, suggesting that transfected cells from the skin migrated to the lymph nodes and expressed the gene encoded by the plasmid DNA. In contrast, less than 4,000 copies of gag mRNA per 10⁶ cells was found in the lymph nodes of mice transduced with the pGFP. This value was similar to that found in several untreated control mice (average of 4,536; SD=1,610). We suspect that the gag-specific RT-PCR reaction gives a high background in the murine model probably due to an interference with endogenous retroviruses that are common in mice.

In order to visualize the transduced cells in the lymph nodes, in situ hybridization experiments were carried out. PEIm/DNA complex encoding the neomycin-phosphotransferase gene (Neo) was placed on the surface of the skin. One day later lymph nodes were removed, fixed and analyzed by in situ hybridization using a P³² labeled Neo-specific antisense probe. FIG. 8a and b show the Neo-expressing cells migrated into the lymph node. Some RNA expressing cells located outside the node, representing cells in the process of entering into the lymph node. Other cells expressing the Neo-RNA were located inside the node, suggesting that they have already entered into the cortex of the lymph node. In situ hybridization of parallel sections using a control probe (sense Neo) did not reveal positive cells (not shown). These results confirmed the RT-PCR data and showed that large numbers no of cells expressing the plasmid DNA-derived RNA accumulated in the draining lymph nodes after application of the PEIm/DNA complex on the surface of the skin.

Protein expression in the lymph nodes after transcutaneous gene transfer. In order to detect protein expression, we analyzed the lymph nodes with an HIV Gag-specific antibody (KC57 FITC, Coulter, Fla.) 72 hours after application of the PEIm/DNA complex on the surface of the skin (FIG. 8c). Positive cells were mainly located in the paracortical area, which is also referred to as the T cell area known to contain the migrating LC-derived DC. Quantification of these cells revealed an average of 222 (30-400) Gag expressing cells per 0.05 mM sections, corresponding to an average of 68 positive cells/mm². Parallel sections, stained with the isotype control showed an average of 0.6 (0-2) positive cells per sections. Altogether, these results demonstrate an unprecedented level of migration and gene expression of DC in the lymph nodes after application of the PEIm/DNA complex on the surface of the skin.

To determine if gentle exfoliation of the skin surface will improve the efficiency of gene transfer, we lightly rubbed the surface of the skin after shaving. This induced a slight redness of the skin. Both shaved and rubbed and only shaved mice were treated simultaneously with the PEIm/DNA complex and inguinal lymph nodes were analyzed by immunohistochemical staining for the Gag antigen. In the lymph nodes of mice that were only shaved we found an average of 338 HIV-1 Gag expressing cells per 9.7 mm² of lymph node section (average: 35 positive cells/mm²). The lymph node of the shaved and rubbed mice revealed an average of 1,138 HIV-1 Gag expressing cells per 13.0 mm² (average 88 positive cells/mm²). Parallel sections, stained with the isotype control resulted in an average of 1 positive cell per mm² in both cases. These results indicate that transcutaneous PEIm/DNA complex-mediated gene transfer to lymph node cells can be improved by slight exfoliation of the stratum corneum.

Transcutaneous gene transfer to dendritic cells in primates. Most of the in vivo LC migration studies performed previously utilized the widely accessible murine model. However, the murine epidermis differs from the human one. For example, mice have dendritic epidermal T cells that produce various cytokines, but these cells have not been found in the human epidermis (Matsue, H., Bergstresser, P. R. & Takashirma, A. Reciprocal cytokine-mediated cellular interactions in mouse epidermis: promotion of gamma delta T-cell growth by IL-7 and TNF alpha and inhibition of keratinocyte growth by gamma IFN. J Invest Dermatol 101, 543-548 (1993); Foster, C. A. et al. Human epidermal T cells predominantly belong to the lineage expressing alpha/beta T cell receptor. J Exp Med 171, 997-1013 (1990)). Thus, we used a rhesus macaque model to determine the efficacy of transcutaneous gene transfer to DC in primates.

The DNA/PEIm complex was applied on the medial thigh of a rhesus macaque. One day later a draining lymph node was surgically removed and gene expression was assayed by in situ hybridization as described in the mice experiment (FIG. 9a). This analysis demonstrated gene-expressing cells located in the T cell area of the lymph node (Steinman, R. M., Pack, M. & Inaba, K. Dendritic cells in the T-cell areas of lymphoid organs. Immunol Rev 156, 25-37 (1997)), specifically in the paracortical region. Some of these cells had already interdigitated into the T cell area. The identity of these positive cells was demonstrated with an antibody specific for lymph node DC (anti-human Fascin, 55K-2, Dako Corp. CA) (Steinman, R. M., Pack, M. & Inaba, K. Dendritic cells in the T-cell areas of lymphoid organs. Immunol Rev 156, 25-37 (1997)) (FIG. 9b). Control hybridization of a parallel section with the sense probe did not detect positive cells. Quantitative analysis revealed 153 DC expressing DNA per 13.4 mm² total analyzed sections (average 11 positive cells/mm²). These results indicate efficient in vivo transduction of DC using this novel transcutaneous DNA immunization technology in non-human primates.

Induction of potent T cell-mediated immune responses in primates. To study the immunogenicity of transcutaneous PEIm/DNA immunization we used a rhesus macaque model and a plasmid DNA derived from the pathogenic SHIV (Karlsson, G. B. et al. Characterization of molecularly cloned simian-human immunodeficiency viruses causing rapid CD4⁺ lymphocyte depletion in rhesus monkeys. J Virol 71, 4218-4225. (1997); Reimann, K. A. et al. A chimeric simian/human immunodeficiency virus expressing a primary patient human immunodeficiency virus type 1 isolate env causes an AIDS-like disease after in vivo passage in rhesus monkeys. J Virol 70, 6922-6928. (1996)). SHIV represents a hybrid genome with the gag and pol genes of SIV_{mac239} and the envelope gene of HIV_{89.6p}. We have constructed a plasmid DNA encoding a replication and integration defective SHIV vector (p_{SHIV}(int-)) and named the PEIm/DNA/glucose composition as DermaVir_{SHIV}. The DNA of DermaVir_{SHIV} was designed to express all SHIV proteins similar to the wild type except the integrase. We mutated the integrase gene because (i) integrase-mutant retroviruses are not only replication defective but also unable to introduce permanent genetic modifications (Stevenson, M., Stanwick, T. L., Dempsey, M. P. & Lamonica, C. A. HIV-1 replication is controlled at the level of T cell activation and proviral integration. Embo J 9, 1551-1560 (1990); Naldini, L., Blomer, U., Gage, F. H., Trono, D. & Verna, I. M. Efficient transfer, integration, and sustained long-term expression of the transgene in adult rat brains injected with a lentiviral vector. Proc Natl Acad Sci U S A 93, 11382-11388 (1996)) and (ii) replication-defective retroviruses have been previously used safely for delivering therapeutic genes in experimental human gene therapy protocols (Riddell, S. R. et al. T-cell mediated rejection of gene-modified HIV-specific cytotoxic T lymphocytes in HIV-infected patients. Nat Med 2, 216-223 (1996); (Morgan, R. A. & Walker, R. Gene therapy for AIDS using retroviral mediated gene transfer to deliver HIV-1 antisense TAR and transdominant Rev protein genes to syngeneic lymphocytes in HIV-1 infected identical twins. Hum Gene Ther 7, 1281-1306 (1996)). Retroviruses, like HIV, have naturally evolved toward efficient expression in the host's cells. We chose to present most of the viral proteins within an authentic expression system because efficient gene expression is important for the induction of potent SHIV-specific immune responses. Indeed, viral genes expressed from a foreign promoter (e.g. CMV) do not express efficiently in primates and do not induce potent immune responses in the absence of humanization (Barouch, D. H. & Letvin, N. L. DNA vaccination for HIV-1 and SIV. Intervirology 43, 282-287 (2000); Corbet, S. et al. Construction, biological activity, and immunogenicity of synthetic envelope DNA vaccines based on a primary, CCR5-tropic, early HIV type 1 isolate (BX08) with human codons. AIDS Res Hum Retroviruses 16, 1997-2008. (2000)). An authentic expression is not only efficient but also results in an authentic processing of viral epitopes. This might be advantageous for the induction of HIV/SIV-specific T cell immunity, because immune control of HIV/SIV has been induced only by replication competent (wild type or attenuated) viruses (Lori, F. et al. Control of SIV rebound through structured treatment interruptions during early infection. Science 290, 1591-1593. (2000); Lisziewicz, J. et al. Control of HIV despite the discontinuation of antiretroviral therapy. N Engl J Med 340, 1683-1684 (1999); Rosenberg, E. S. et al. Immune control of HIV-1 after early treatment of acute infection. Nature 407, 523-526 (2000); Daniel, M. D., Kirchhoff, F., Czajak, S. C., Sehgal, P. K. & Desrosiers, R. C. Protective effects of a live attenuated SIV vaccine with a deletion in the nef gene. Science 258, 1938-1941 (1992); Rowland-Jones, S. et al HIV-specific cytotoxic T-cells in HIV-exposed but uninfected Gambian women. Nat Med 1, 59-64 (1995); Almond, N. et al. Protection by attenuated simian immunodeficiency virus in macaques against challenge with virus-infected cells. Lancet 345, 1342-1344 (1995)).

Four macaques were immunized with DermaVir_{SHIV} on approximately 2.5cm² surface of the skin on four locations: the left and right upper inner thigh and left and right brachial area. A slight skin rash developed due to the skin preparation that resolved completely within days. 0.2 mL of DermaVir_{SHIV} containing 0.025 mg DNA was applied per location for 45 min. DermaVir_{SHIV} immunization had no toxic side effects.

SIV- and HIV-specific CD8⁺ T cells in the peripheral blood were quantified by CD8VIR assay (Lori, F. et al. Control of SIV rebound through structured treatment interruptions during early infection. Science 290, 1591-1593. (2000)). CD8VIR quantifies all virus specific CD8⁺ T cells that can respond to SIV/HV stimulation, which results in IFN-γ production. Four experimental animals were tested before (naive macaques, Table 5) and 3 weeks after (DermaVir immunized macaques, Table 5).

TABLE 5
SIV- and HIV-specific T cell responses
in DermaVir_{SHIV} immunized rhesus macaques.

| | | | | | |
|---|---|---|---|---|---|
| Stimula- | No | HIV | SIV | | |
| tion | Antigen | antigen | antigen | CD8VIR_{HIV} | CD8VIR_{SIV} |

Naïve macaques

| | | | | | |
|---|---|---|---|---|---|
| 1 | 1,100 | 700 | 800 | 0 | 0 |
| 2 | 1,000 | 1,000 | 900 | 0 | 0 |
| 3 | 4,100 | 4,200 | 700 | 100 | 0 |
| 4 | 3,600 | 2,800 | 1,000 | 0 | 0 |
| Average | 2,450 | 2,175 | 850 | 25 | 0 |
| SD | 1,630 | 1,638 | 129 | 50 | 0 |

DermaVir immunized macaques

| | | | | | |
|---|---|---|---|---|---|
| 1 | 3,700 | 2,600 | 11,000 | 0 | 7,300 |
| 2 | 1,900 | 3,000 | 6,100 | 1,100 | 4,200 |
| 3 | 3,000 | 2,800 | 5,500 | 0 | 2,500 |
| 4 | 3,100 | 3,000 | 4,300 | 0 | 1,200 |
| Average | 2,925 | 2,850 | 6,725 | 275 | 3,800 |
| SD | 750 | 191 | 2,947 | 550 | 2,637 |

Values represent the numbers of IFN-γ expressing CD8⁺ T cells per 10⁶ CD8⁺ T cells. CD8VIR are calculated as differences between the number of IFN-γ positive cells after SIV/HIV-specific activation and the number of IFN-γ positive cells with mock (no antigen) activation.

DermaVir_{SHIV} immunization. Representative histograms are shown in FIG. 10. CD8VIR was undetectable prior to DermaVir_{SHIV} vaccination in the experimental animals. Three weeks after DermaVir_{SHIV} immunization all the animals developed SIV-specific T cell responses. In the peripheral blood we found an average of 3,800 SIV-specific CD8⁺ cells per 10⁶ CD8⁺ cells. Activation of a high amount of SIV-specific T cells by DermaVir_{SHIV} was expected because the gag and reverse transcriptase proteins of SHIV vector are derived from SIV and these proteins are known to be highly immunogenic. DermaVir_{SHIV} could potentially induce HIV-specific immune responses, because the SHIV vector encodes an HIV-1 envelope gene. However HIV-specific cells were undetectable in three of the four immunized animals. This might be due to the poor immunogenicity of the envelope gene and/or the absence of cross-reactive epitopes between the SHIV vector envelope (HIV_{89.6p}) and the HIV envelope (HlV_{MN}) used to measure the VIR responses. These results demonstrated that transcutaneous DermaVir_{SHIV} immunization could induce potent T cell-mediated immune responses in non-human primates.

Discussion

We have described a novel technology for induction of T cell-mediated immune responses that is based on the in vivo transfer of DNA to epidermal LC that migrated to the lymph node. DNA/PEIm complexes were applied on the surface of the skin. Large granular cells in the skin picked up the DNA, migrated from the epidermis to the culture media, and expressed the DNA-encoded antigen in vitro. The distinct large, granular cells that moved out from the skin to the culture media have been previously characterized as LC (Larsen, C. P. et al. Migration and maturation of Langerhans cells in skin transplants and explants. J Exp Med 172, 1483-1493 (1990); Larsen, C. P. & Austyn, J. M. Langerhans cells migrate out of skin grafts and cultured skin: a model in which to study the mediators of dendritic leukocyte migration. Transplant Proc 23, 117-119 (1991)). Epithelial cells located in the epidermis are not able to pick up the mannosylated complexes and do not migrate to the culture medium or to the lymph nodes (Pope, M. et al. Dendritic cell-T cell conjugates that migrate from normal human skin are an explosive site of infection for HIV-1. Adv Exp Med Biol 378, 457-460 (1995)). Similar complexes have been previously shown to target the mannose receptors located on immature DC in vitro (Diebold, S. S., Kursa, M., Wagner, E., Cotten, M. & Zenke, M. Mannose polyethylenimine conjugates for targeted DNA delivery into dendritic cells. J Biol Chem 274, 19087-19094 (1999)).

As epidermal LC matured into DC and migrated into the lymph nodes, both mRNA and protein encoded by the DNA were expressed in the draining lymph nodes. Our experiments demonstrated a remarkably high efficiency of in vivo gene transfer. In the mouse model the efficacy was between 30-90 cells/mm² and in the primate model 11 cells/mm². The disparity of these numbers might reflect the differences between the murine and primate epidermis. The average diameter of a lymph node is about 1.8 mm and 6 mm in mice and monkeys, respectively. When the size of the lymph node was taken into account, a conservative estimate of the total number of positive cells per lymph node in the murine model was between 1,000 and 4,000, whereas in the primate model was about 20,000 cells.

How could a non-invasive method transduce so many DC in the lymph nodes? After epidermal LC pick up the PElm-DNA complexes they might receive a signal to migrate from either the mannosylated complex or by the cytokines secreted by nearby keratinocytes, or both. These “danger” signals are thought to trigger the LC to leave the epidermis and migrate via the lymphatic vessels (veiled cells) to the draining lymphoid organs (Dieu-Nosjean, M. C., Vicari, A., Lebecque, S. & Caux, C. Regulation of dendritic cell trafficking: a process that involves the participation of selective chemokines. J Leukoc Biol 66, 252-262 (1999)). During the migration, the PEIm could buffer the endosomes, liberate the complex into the cytoplasm (Boussif, O. et al. A versatile vector for gene and oligonucleotide transfer into cells in culture and in vivo: polyethylenimine. Proc Natl Acad Sci USA 92, 7297-7301 (1995)) and facilitate the trafficking of the DNA into the nucleus, where the DNA-encoded antigens are expressed (Pollard, H. et al. Polyethylenimine but not cationic lipids promotes transgene delivery to the nucleus in mammalian cells. J Biol Chem 273, 7507-7511 (1998)). The cells containing the DNA in the lymphoid organs eventually settled in the T cell reach area as interdigitating DC. In the monkeys, the DermaVir_{SHIV} was applied to a total area of about 20 cm² of skin, which could contain about 2×10⁶ LC. These LC are all potential targets for gene transfer. Since we found about 20,000 DNA expressing cells in the lymph nodes, we estimated that at least one out of 100 transduced LC reached the draining lymph node and expressed the DNA. We can conservatively assume that all nodes (6-8 of them) in the draining area will have similar amounts of positive cells, suggesting that 1-8% of the LC from the skin eventually become antigen-presenting DC. A similar calculation in the mice indicated about 1% in vivo transduction efficacy. Previously described methodologies utilized plasmid DNA introduced into the host either by needle injection in the muscle or skin, or by particle bombardment into the skin with a gene gun. In those instances, the DNA was transferred predominantly to the local somatic cells (e.g. myocytes, keratinocytes and fibroblasts) that served as an antigen reservoir (Tuting, T., Storkus, W. J. & Falo, L. D., Jr. DNA immunization targeting the skin: molecular control of adaptive immunity. J Invest Dermatol 111, 183-188 (1998); Akbari, O. et al. DNA vaccination: transfection and activation of dendritic cells as key events for immunity. J Exp Med 189, 169-178 (1999)). Antigens released by these cells were taken up by DC, processed and presented to T cells. Direct genetic modification of DC in the lymph node occurred only occasionally and was quite inefficient (maximum 30 gene expressing cells per lymph node). The DermaVir technology described here generates unprecedented high levels of gene expression in the lymphoid organs, and is considerably more efficient in generating antigen-presenting DC than other traditional methods of DNA delivery.

In addition to high efficiency transduction in vivo, the formulation of DermaVir developed here offers several advantages: (1) a small size, facilitating the diffusion and penetration via the stratum comeum into epidermal LC; (2) stability at room temperature; (3) protection of plasmid DNA from nuclease degradation by PEIm. Furthermore, (4) the formulation in a physiologically acceptable glucose solution and (5) the needleless application and (6) the low amount of DNA requirement makes this technology suitable for human use.

Since it is well established that DC are very potent inducers of T cell-mediated immune responses we expected that antigen expressing lymph node DC would be capable of priming naive T cells efficiently. Our results demonstrated the activation of large numbers of DNA-encoded antigen-specific CD8⁺ T cells. These results further confirm that this transcutaneous vaccination technology created potent antigen presenting DC in the primate lymph nodes.

The technology described here could provide the basis for novel DNA-based medicines. Various plasmid DNA could be constructed to obtain expression of a wide variety of tumor and viral antigens by DC in the lymph nodes and induce antigen-specific T cell responses. In addition, immune responses might be augmented by the co-expression of recombinant cytokines within the same DNA (Ahuja, S. S. et al Dendritic cell (DC)-based anti-infective strategies: DCs engineered to secrete IL-12 are a potent vaccine in a murine model of an intracellular infection. J Immunol 163, 3890-3897 (1999); Takayama, T., Tahara, H. & Thomson, A. W. Transduction of dendritic cell progenitors with a retroviral vector encoding viral interleukin-10 and enhanced green fluorescent protein allows purification of potentially tolerogenic antigen-presenting cells. Transplantation 68, 1903-1909 (1999); Ozawa, H. et al. Granulocyte-macrophage colony-stimulating factor gene transfer to dendritic cells or epidermal cells augments their antigen-presenting function including induction of anti-tumor immunity. J Invest Dermatol 113, 999-1005 (1999); Melero, I. et al. Intratumoral injection of bone-marrow derived dendritic cells engineered to produce interleukin-12 induces complete regression of established murine transplantable colon adenocarcinomas. Gene Ther 6, 1779-1784 (1999)). Genetic manipulation of DC to express IL-10, TGF-beta, FasL and CTLA4Ig has also been suggested to enhance tolerance and allograft survival (Lu, L. et al. Genetic engineering of dendritic cells to express immunosuppressive molecules (viral IL-10, TGF-beta, and CTLA4Ig). J Leukoc Biol 66, 293-296 (1999)). In addition, the techniques described herein might be used as tools to elucidate as yet unanswered questions in immunology, such as the role of lymph node DC in antigen presentation and immune induction.

13. A Therapeutic Vaccine

We have previously shown that fixed-scheduled STI-HAART induces control of virus replication during drug treatment interruptions in SIV₂₅₁-infected monkey treated early after infection (Lori, F. et al. Control of SIV rebound through structured treatment interruptions during early infection. Science 290, 1591-1593. (2000)). Similar data has been obtained in HIV-1 infected patients treated early after infection (Lisziewicz, J. et aL Control of HIV despite the discontinuation of antiretroviral therapy. N Engl J Med 340, 1683-1684 (1999); Rosenberg, E. S. et al. Immune control of HIV-1 after early treatment of acute infection. Nature 407, 523-526 (2000)). These findings suggested that controlled rebounds of HIV-1 might be utilized for auto-vaccination in acutely infected individuals.

To compare continuous HAART with fixed scheduled STI-HAART (Lori, F. et al. Control of SIV rebound through structured treatment interruptions during early infection. Science 290, 1591-1593. (2000)) during AIDS, a cohort of ten SIVmac₂₅₁-infected (for 14 months) rhesus monkeys was enrolled in a randomized study. Disease progression in SIVmac₂₅₁-infected rhesus monkeys is faster that in HIV-1-infected humans. Uninfected animals have high CD4 counts (ca. 2,000 counts/mm³) and AIDS occurs generally after one year of infection when ca. 70% of the animals die. The study cohort originated from a larger cohort of concurrently infected animals in which 14 died prior to the study. During the protocol approval period three additional monkeys died. Unfortunately, comparable animals were not available to increase the size of the cohort. The remaining seven monkeys were randomized such that three animals received continuous HAART and four animals STI-HAART (the first two treatment cycles were 4 weeks on drugs and three weeks off then 3 weeks on and 3 weeks off, as previously described (Lori, F. et al. Control of SIV rebound through structured treatment interruptions during early infection. Science 290, 1591-1593. (2000))).

HAART successfully suppressed virus replication (median viral load <200 copies/mL) and improved the CD4 counts (median CD4 increase 498 cells/mm³) in all the animals enrolled in the continuous HAART group after 133 days of treatment (FIG. 11a). Then, two animals spontaneously experienced a viral rebound (due to the development of PMPA and ddI resistant virus) and/or a sharp loss of CD4. Their clinical conditions deteriorated, and both animals were sacrificed at day 183. The third animal died at day 224, due to drug related toxicity (diabetes). The evolution of the disease in these animals was similar to what is observed in AIDS patients treated with HAART. (Sansone, G. R. & Frengley, J. D. Impact of HAART on causes of death of persons with late-stage AIDS J Urban Health 77, 166-75. (2000)) That is, initial response to therapy, followed by viral break-through, and final death because of AIDS and/or drug related toxicity.

HAART also suppressed viral load in animals enrolled in the STI-HAART arm (FIG. 11b). In contrast to animals treated early after infection (Lori, F. et al. Control of SIV rebound through structured treatment interruptions during early infection. Science 290, 1591-1593. (2000)), the animals with AIDS did not control virus rebound during the treatment interruptions; however, viral load was suppressed after each therapy re-start. Concomitant with the rebound of viral load CD4 counts declined, and then increased after treatment re-start. Comparable to HAART, STI-HAART resulted in a significant increase of CD4 count 133 days after treatment initiation (median CD4 increase 436 cells/mm³). One of three animals died of AIDS in the STI-HAART group, 9 months after treatment initiation, presumably due to the virus rebound, which was extremely high. The better survival rate among animals treated with STI-HAART is consistent with the results obtained with acutely infected animals (Lori, F. et al. Control of SIV rebound through structured treatment interruptions during early infection. Science 290, 1591-1593. (2000)). HIV-infected patients with chronic infection also fail to control virus rebounds and CD4 counts decrease during treatment interruptions, but recover both CD4 counts and viral suppression after therapy is reinitiated (Carcelain, G., et al. Transient mobilization of human immunodeficiency virus (HIV)-specific CD4 T-helper cells fails to control virus rebounds during intermittent antiretroviral therapy in chronic HIV type 1 infection J Virol 75, 234-41. (2001)). These results further confirm the similarity between clinical evolution of the disease in animals and in humans.

The three animals in the STI-HAART group continued the STI-HAART cycles. The viral load rebound persisted during the six consecutive treatment interruption cycles (FIG. 12a), suggesting that additional STI-HAART cycles could not improve the control of virus replication during treatment interruptions. To the contrary, nine months after treatment initiation the median viral load during the last treatment interruption had increased to U.S. Pat. No. 4,292,260 copies/mL. In addition, in one animal (#60), re-initiation of HAART failed to suppress the viral load. To study potential benefit of a vaccination, we decided to treat these animals with a novel therapeutic vaccine called DermaVir_{SHIV}. This vaccine was designed to initiate neoteric SHIV-specific T cell immunity. Virus-specific T cell responses have been previously shown to control virus replication in some long-term non-progressors (Rosenberg, E. S., LaRosa, L., Flynn, T., Robbins, G. & Walker, B. D. Characterization of HIV-1-specific T-helper cells in acute and chronic infection Immunol Lett 66, 89-93. (1999)) or during drug treatment interruption early after infection (Lori, F. et al. Control of SIV rebound through structured treatment interruptions during early infection. Science 290, 1591-1593. (2000); Lisziewicz, J. et al. Control of HIV despite the discontinuation of antiretroviral therapy. N Engl J Med 340, 1683-1684 (1999); Rosenberg, E. S. et al. Immune control of HIV-1 after early treatment of acute infection. Nature 407, 523-526 (2000)). However, it was challenging to see whether DermaVir_{SHIV} could be beneficial during late stage AIDS, when the immune system is believed to be destroyed.

DermaVir_{SHIV} is a glucose-water solution containing a plasmid DNA as an active ingredient and polyethylenimine-mannose (PEIm) as an adjuvant (See Example 12). One therapeutic application contained 0.1 mg DNA capable of expressing all but the integrase protein of the Simian-Human Immunodeficiency Virus (SHIV). DermaVir_{SHIV} was formulated to transduce Langerhans cells located in the epidermis and it was applied on the surface of the skin of the animals. We have shown that these Langerhans cells are triggered to migrate to the lymph nodes, mature to dendritic cells and present SHIV antigens to naive T cells. After SHIV-specific activation of naive T cells in the lymph nodes, DermaVir_{SHIV} initiated potent SIV-specific T cell-mediated immune responses in uninfected monkeys (See Example 12).

The STI-HAART study was amended to administer two medications of DermaVir_{SHIV} in combination with HAART during the treatment cycles (10 and 3 days before treatment interruptions). The treatment schedule and the median viral load changes are shown in FIG. 12b. During the first therapy interruption following the DermaVir_{SHIV} treatment the median viral rebound was 12,000 copies/ml. The magnitude of this rebound was unexpected, since it was >2 log lower than observed during the previous interruption (U.S. Pat. No. 4,292,260 copies/ml). During the next interruption the magnitude of viral load rebound further decreased from 12,000 to 460 copies/mL. Finally, during the last treatment interruption, the median viral load remained under the limit of detection (>200 copies/mL).

The animals did not experience any toxic side effects associated with the DermaVir_{SHIV} medication. The skin became slightly red due to the exfoliating preparation of the skin, but this local irritation disappeared within 2 days. Interestingly, four weeks after the first DermaVir_{SHIV}-STI-HAART cycle, all the animals had enlarged lymph nodes near the vaccination sites. One week later, most of the lymph nodes were back to normal size. We suspect that the enlarged draining lymph nodes represented a sign of the successful initiation of T cell responses. This is consistent with our results indicating that Langerhans cells carry very efficiently antigens to the draining lymph nodes after DermaVir_{SHIV} vaccination (See Example 12).

The dynamics of viral rebound in each animal were also interesting. During the first therapy interruption after HAART-DernaVir_{SHIV} treatment, two animals (#51, #56) had partially controlled viral rebound even before therapy was re-initiated (FIG. 13a,b). Viral rebound further decreased during the next treatment interruptions. It remained undetectable in one animal (#56) after the second, and in the other animal (#51) after the third treatment interruptions. The viral load of monkey #56 had remained undetectable from the second interruption cycle until the following 90 days (FIG. 13b). Although in the third animal (#60) a virus rebound was consistently observed during treatment interruptions probably because of the onset of a drug-resistant mutant, the extent of the rebound progressively decreased (FIG. 13c).

The CD4 counts fluctuated during DermaVir_{SHIV}-STI-HAART. Generally, CD4 count increased after the treatment and decreased with viral rebound (FIG. 13a, b, c). The highest CD4 count variations were detected in animal #60, who had the highest viral load rebounds (FIG. 13c). Interestingly, animal #56, which first achieved control of viral load during treatment interruptions, had the lowest CD4 count compared to the other two animals (FIG. 13b). A low number of CD4 T lymphocytes might contribute to the control of the virus production during the treatment interruption cycles. Similar conclusions have been reached during an induction/maintenance therapy clinical trial where loss of viral suppression was associated with greater increases of CD4 counts. (IIvlir, D. V., et al. Maintenance Antiretroviral Therapies in HIV-Infected Subjects with Undetectable Plasma HIV RNA after Triple-Drug Therapy N Engl J Med 339, 1261-1268. (1998)).

The comparison of the rate of viral load rebound among those animals undergoing STI-HAART early after infection (Lori, F. et al Control of SIV rebound through structured treatment interruptions during early infection. Science 290, 1591-1593. (2000)), those initiating STI-HAART during AIDS, and the same animals treated with STI-HARRT plus DermaVir_{SHIV} revealed an interesting pattern. The rate of viral rebound during consecutive HAART interruptions, that was unchanged before the initiation of vaccine therapy, decreased sharply after vaccination, and became remarkably similar to that observed in the animals treated with STI-HAART early after infection (FIG. 14). These results suggest that DermaVir_{SHIV} therapy can improve the control of virus replication during interruption of HAART.

The unexpected therapeutic efficacy of DermaVir_{SHIV} in animals at late stage of the disease reveals a previously unsuspected capacity of the host to respond to vaccination and provides new optimism that the induction of virus control might still be achievable long time after the initial retroviral infection. These results warrant further investigation of DermaVir and possibly other therapeutic vaccination approaches in HIV-infected individuals at different stages of the disease.

What is claimed is:

1. A method of therapeutic genetic immunization, comprising administering an antiretroviral drug therapy until viral replication is effectively suppressed, and then administering a gene delivery complex comprising foreign genetic material and a non-viral vector, wherein the complex has a specific affinity for a receptor on an antigen presenting cell.

2. The method of claim 1, wherein the foreign genetic material is selected from the group consisting of RNA and DNA.

3. The method of claim 1, wherein the foreign genetic material encodes at least one antigen from a reverse-transcriptase dependent virus or a mutant reverse-transcriptase dependent virus.

4. The method of claim 3, wherein the foreign genetic material is DNA encoding at least a substantial portion of a replication-defective human immunodeficiency virus.

5. The method of claim 3 or 4, wherein the foreign genetic material is DNA encoding at least a substantial portion of an integration-defective human immunodeficiency virus.

6. The method of claim 5, wherein the foreign genetic material is DNA encoding at least a substantial portion of an integrase negative mutant of a dual-tropic primary isolate of a human immunodeficiency virus.

7. The method of claim 6, wherein the DNA further includes one or more stop codons in one or more of the reading frames of the integrase gene.

8. The method of claim 1, wherein the complex is DNA and one or more agents selected from the group consisting of sugars, polyethylenimine, polyethylenimine derivatives, and mixtures thereof.

9. The method of claim 8, wherein the agent is sugar-modified polyethylenimine.

10. The method of claim 8, wherein the agent is glucose.

11. The method of claim 8, wherein the complex has a specific affinity for the mannose receptor.

12. The method of claim 1, wherein the antigen presenting cell is a Langerhans cell.

13. The method of claim 1, wherein the antigen presenting cell is a dendritic cell.

14. The method of claim 13, wherein the receptor is a mannose receptor.

15. The method of claim 1, wherein the antiretroviral drug therapy comprises an effective amount of hydroxyurea and a reverse transcriptase inhibitor.

16. The method of claim 15, wherein the reverse transcriptase inhibitor is selected from ddI, d4T, 3TC, AZT, delavirdine, abacavir, adefovir, nevirapine, efavirenz, lubocavir PMPA PMEA, and mixtures thereof.

17. The method of claim 1, wherein the antiretroviral drug combination comprises a highly active retroviral drug therapy.

18. The method of claim 17, wherein the drug combination is one or more reverse transcriptase inhibitors, and one or more protease inhibitors.

19. The method of claim 18, wherein the reverse transcriptase inhibitor is selected from ddI, d4T, 3TC, AZT, delavirdine, abacavir, adefovir, nevirapine, efavirenz, lubocavir, PMPA PMEA, and mixtures thereof.

20. The method of claim 18, wherein the protease inhibitor is selected from indinavir, saquinavir, ritonavir, nelfinavir, GW141, and mixtures thereof.

21. The method of any one of claims 17-20, wherein the highly active antiretroviral drug therapy further comprises hydroxyurea.
